(19) United States
(12) Patent Application Publication
(10) Pub. No.: US 2019/0099592 A1
(43) Pub. Date: Apr. 4, 2019

(54) DISINFECTING INTRAVENOUS CONNECTORS

(71) Applicant: CK Forward, LLC, Menasha, WI (US)

(72) Inventor: William Christopher Kwasny, Menasha, WI (US)

(73) Assignee: CK Forward, LLC, Menasha, WI (US)

(21) Appl. No.: 15/724,465

(22) Filed: Oct. 4, 2017

(51) Int. Cl. A61M 39/16; A61M 39/10; A61L 2/16

(52) U.S. Cl. CPC A61M 39/162; A61M 39/1011; A61M 39/165; A61J 1/1443; A61M 2039/1088; A61M 2209/088; A61L 2/16

(57) ABSTRACT

Disinfecting nubs, disinfecting nub assemblies, and methods of use for disinfecting medical access ports, and maintaining such ports disinfected. In nub assemblies, nubs are mounted on substrates. Substrates can have substrate connectors, used to connect the nub assembly to an object. That object can be a user's body or on an IV tubing assembly. Mounting the nub assembly on the user's body means nubs are available wherever needed, at multiple user locations. Mounting substrate connectors to IV tubing connected to a patient, especially mounted adjacent a medical access port, such as a luer connector, on that tubing, makes the nubs immediately available when and where needed for disinfecting that port. The disinfecting nub can be squeezable. Dead fold properties of the nub hold the nub on the port after the port is inserted into the nub, the nub is squeezed, and the squeezing is released.

### BACKGROUND OF THE INVENTION

In the medical profession, it is common to administer one or more liquid medications to a patient intravenously. To accomplish this, a needle, carrying a surrounding catheter, is typically inserted into a vein. Once the needle has been inserted, the needle is withdrawn and the catheter is left as the point of patient entry and connected to intravenous fluid tubing.

Once the intravenous tubing has been connected to the patient at the catheter, use of one or more medical access ports enables a medical professional to quickly connect a particular source of fluid/medication, at an opposing end of the tubing, or at an intermediate portion of the tubing, to the intravenous tubing which is connected with the patient blood vessel, thus to administer the fluid/medication to the patient. Medications can then be introduced into the intravenous tubing through such medical access ports for administration into the patient's bloodstream.

Various valves, Y-connectors, medical access ports, and the like may be used, in combination with the intravenous tubing, to connect the patient, through one or more ports, to respective one or more medication sources. Such medication sources may be bags or vials of fluid product, needled or needle-less syringes, or other needle-less containers. Such intravenous tubing is commonly pre-packaged as a tubing assembly, provided with connectors, ports, valves and the like which are known to be commonly used in a given procedure.

The needle-less connection between the intravenous tubing and a medication source can be what is commonly referred to as a “luer” connection, and the respective connectors which are used to make such connections are referred to as “luer connectors”. “Luer” is a generic term used in the art to refer generally to specific products which are used to make such connections, and which are also known as, for example and without limitation, needle-less connectors, male luer connectors, female luer connectors, catheter hubs, and luer hubs. Poke-through ports are also available in some intravenous tubing assemblies for use with needled syringes.

In addition, where first and second sections of intravenous tubing are connected to each other in series, luer-type connectors can be used in making the connection.

The term “luer”, referring to tubing connectors, is well known in the medical field and is used here to mean mating structures, with or without threads, which allow two mating devices, namely luer-compatible components, to be joined to each other for fluid communication.

The intravenous tubing assembly can include a number of connectors at the end of the tubing, or intermediate the length of the tubing, providing opportunity for connections to medication sources. Between the time a tubing assembly has been removed from its packaging and the time when a given connector is connected to a medication source, that connector is susceptible to being contaminated with pathogen organisms from the air and from incidental contact with objects such as the patient, a healthcare provider, or other object in the vicinity of the procedure.

A particular health care problem has been recognized in recent years, as healthcare-associated infections, known in the medical field as “HAI's”. Although it is usually difficult to determine the exact source of a post operative wound infection, there is growing evidence that contaminated intravenous line connectors can lead to HAI's. Recent studies have used molecular typing techniques to show that bacteria found in the operating room and on intravenous connectors matched the bacterial types found in subsequent infections.

While the rate of infections might seem low to some, the end results of such health care associated infections is sobering, as the death rate is very high, the cost of treating such infections is very high, and the legal ramifications to the health care profession in terms of professional liability insurance, are costly.

Various efforts are being made to reduce the exposure of patients to infectious organisms during medical treatments, thus to reduce HAI's. Namely, sterilization and various cleaning procedures, as well as other care improvements, have become standard practices in healthcare environments. One such effort has been, and remains, to take steps to disinfect connectors on the intravenous fluid tubing, as well as rubber access ports on vials of liquid medication which are being used. To that end, standard practice teaches that a healthcare professional should assure that every connector is disinfected before being accessed by a needle or connected to an intravenous tubing assembly/line. As part of that effort, the healthcare profession teaches that one should assume that every medical access port, which is not connected to an intravenous tubing or syringe, or which becomes disconnected from such intravenous tubing or syringe, is contaminated, and should correspondingly be disinfected before that connector is re-connected with the intravenous tubing assembly.

While the principle of uniform disinfection is well known, consistency in the practice is less than desired.

The old standard in the medical practice takes the form of a small disinfectant wet wipe which is a nonwoven substrate impregnated with a disinfectant material. Each wet wipe is individually packaged in a small sealed pouch. To disinfect a given medical access port, the practitioner accesses a pouch containing a wipe, tears open the pouch, accesses the wet wipe, and uses the wet wipe to scrub the respective connector, scrubbing both on the end of the connector and on the sides of the connector.

A number of problems are associated with such wet wipes. A first problem is that opening the wet wipe pouch requires use of two hands. Such wet wipes are difficult for some users to grip, especially when wearing gloves, because the wet wipes are so small. The wet wipes can easily be dropped. And a supply of the wet wipes may not be present at the use site; or the user may move to a different work location where wet wipes are not available.

The healthcare profession has addressed “unused” connectors by providing a variety of disinfecting caps which can be removably mounted to an unused connector until such time as use of the connector is needed for administration of a medication. Namely, a connector which is not currently being used may be inserted into the openable end of such cap.

Such caps generally take the form of a rigid plastic receptacle having a closed end and an openable end. A disinfectant material is contained in the receptacle. The disinfectant material commonly consists of a substrate such as a non-woven sheet, a foam, or the like, saturated with a disinfectant liquid. The cap may be threaded for mounting to a locking luer connector. Or the cap may be threadless for mounting to a slip connector. In either case, the end of the unused connector is inserted into the cap whereupon the disinfectant material comes into contact with the connector and is expected to disinfect the end surface and the side surfaces, of the connector. As long as the connector remains inside the cap, the connector is considered sterile and can be connected, without further disinfection procedure, to a mating connector for connecting the tubing assembly to a medication source. However, any time the connector is removed from the cap and left exposed to ambient conditions for any significant period of time, the connector needs to be again disinfected before being connected to a fluid source or destination.

Depending on the healthcare setting, and patient needs, a given connector may stay connected to a given medication source for an extended period of time. In the alternative, as patient needs change, it may be appropriate for a healthcare professional to disconnect a given medication from the patient whereupon the respective luer connector or other medical access port is exposed to ambient conditions, which provide opportunity for pathogenic contamination of the respective connector. The healthcare professional then has a decision to make. Does he/she immediately access a disinfecting cap and insert the now-disconnected connector into the cap? Or does the healthcare professional leave the connector exposed and presume to disinfect the connector before re-connecting the connector to the next medication to be administered through that specific connector?

In a hospital ward setting, intensive care unit, operating room, or other patient treatment area, a patient may have several unused connectors on a tubing assembly being used to administer medication at any given time. Also, a given connector may be used intermittently. The timing for the next use of a given connector to administer a medication may be unknown, and/or unknowable. Generally, decisions regarding capping a connector are made by very busy medical professionals. And while installing the connector in a cap takes only a short time, medical personnel are typically stressed for time to complete all assigned activities. Accordingly, medical personnel are motivated to look for activities which can be postponed or eliminated. Historically, the connector has not always been installed in a cap, which leaves opportunity for the connector to become contaminated. The question then becomes whether a medical professional does the needed disinfection before re-connecting the connector to a medication.

In the operating room, an anesthesiologist makes multiple connections to medication throughout an operating procedure. Trade literature reports a typical 6 to 10 changes to the medication through a given connector during a typical operating room procedure. And in some instances, up to 60 to 100 such connections and disconnections may be made. Each time such connection is made, the connector/port is to be disinfected.

Particularly in the operating room, there may be an ongoing demand for the anesthesia provider to respond rapidly to a changing patient need for different medications to be connected at a given connector. In such case, the anesthesia provider needs to act quickly in response to each such need for change in the patient medication, including to disinfect the connector before each such medication is connected to the connector. And where the connector is disconnected for only a short period of time, or where a disinfecting wipe is not readily available at the connector, the anesthesia provider may be tempted to forego disinfecting the connector in order to be able to more quickly make the next connection to medication.

The above-mentioned caps have more recently been proposed, to be used in place of the wet wipes. However, such caps require that the connector be inserted into the cap. And the cap is not conducive to manual scrubbing of the connector. Rather, the literature generally teaches that the connector should be left inserted into the cap for at least a minute. But the patient need may be urgent, not allowing for a minute for disinfecting the connector. At that point, the healthcare professional is caught between the competing demands for rapid response time for administering the medication and the longer time needed to assuredly disinfect the connector.

Accordingly, it would be desirable to provide a wiping and/or scrubbing product which can quickly disinfect the connector, and which is easily accessed.

It would also be desirable to provide such wiping and/or scrubbing product which can be accessed and used with only one hand.

It would further be desirable to provide such wiping and/or scrubbing product which is always present and available at a user location, wherever the user healthcare professional happens to be or to move.

It would further be desirable to provide a such wiping and/or scrubbing product wherein the disinfecting material is contained in a disinfectant carrier in a receptacle, and wherein the containing receptacle enables the user to expel a portion of the disinfectant carrier, and thus a portion of the disinfecting material, from the receptacle, optionally using only one hand, whereby both the disinfecting material and the surface of the connector are visible to the user while the user is using the disinfecting material to scrub the respective connector.

It would also be desirable, in some instances, to provide a plurality of such wiping and/or scrubbing products, mounted to the intravenous tubing assembly at a location where a connector may need to be disinfected multiple times during a given medical procedure.

It would also be desirable to provide a plurality of such wiping and/or scrubbing products, mounted to a substrate, wherein the substrate is mounted and/or restrained to either the user, the user's clothing, or to the tubing assembly, such that such mounting/restraint of the substrate so impedes movement of the substrate as to facilitate releasing a such wiping and/or scrubbing product from the substrate, optionally using only one hand, without need to further restrain the substrate.

It would also be desirable to provide a disinfecting nub wherein the nub can be squeezed about a medical access port, and dead fold properties of the nub retain the nub in a squeezed, and disinfected, condition about the medical access port after the squeezing force has been released.

It would also be desirable to provide methods which facilitate use of such wiping and/or scrubbing products by providing mounting structure which facilitates mounting a strip of multiple such wiping and/or scrubbing products either to the user's body or clothing, or to the tubing assembly adjacent a connector.

It would further be desirable to provide methods wherein a medical access port is inserted into the nub, the nub is then squeezed to exert increased levels of force normal to respective ones of the surfaces of the port, including side surfaces, and to then continue the squeeze while rotating the nub and port relative to each other so as to apply squeeze friction forces to actively scrub respective surfaces of the port.

These, and other, needs are addressed and resolved, or at least attenuated, by the instant invention.

### SUMMARY OF THE INVENTION

The present invention provides a novel disinfecting nub, disinfecting nub assemblies, and methods of use in combination with medical procedures. The use of the disinfecting nub is in disinfecting medical access ports, and maintaining such ports disinfected, thus to assist in preventing healthcare-associated infections. In the nub assemblies, the nubs are mounted on a substrate. Certain of the substrates have substrate connectors which are used to connect the nub assembly to an object

In some instances, that object is the body of a healthcare professional who has use for such disinfecting nubs at more than one location. By providing for the nub assembly to be mountable to the user's body or clothing, the nubs are available wherever needed, for example by an anesthesiologist in the operating room, both at the patient at operating table, and at the medication cart.

In other instances, the nub assembly is configured to be mounted to the intravenous fluid tubing assembly which is connected to the patient being treated, especially mounted adjacent one of the medical access ports, such as a luer connector, on that tubing assembly, thus to be immediately available when and where needed to be used to disinfect the respective medical access port.

In some instances, the disinfecting nub is squeezable and the material of the disinfecting nub has dead fold properties such that, after the medical access port is inserted into the nub and the nub squeezed, and the squeezing is subsequently released, the dead fold properties of the nub retain, hold the medical access port in the nub housing.

In a first family of embodiments, the invention comprehends a disinfecting nub assembly for assisting in preventing healthcare-associated infections, the disinfecting nub assembly, having an assembly length, and comprising a substrate, comprising a substrate body, the substrate body having opposing surfaces, and opposing first and second ends, and a substrate body length between the first and second ends; a plurality of disinfecting nubs mounted to the substrate body, a given disinfecting nub comprising a housing, the housing having an openable end and a closed end, and sidewalls extending between the openable end and the closed end, thus to define a nub cavity inside the housing, a disinfectant carrier being disposed in the nub cavity; and first and second substrate connectors associated with the opposing ends of the substrate body, the first and second substrate connectors having connector lengths extending away from the respective ends of the substrate body, the first and second substrate connectors cooperating with each other such that, when the first and second substrate connectors are wrapped at least part way around an object, the first and second substrate connectors can be connected to each other, thereby to mount the disinfecting nub assembly to the object.

In some embodiments, the connection of the first and second connectors to each other can be adjusted thus to facilitate utility of the disinfecting nub assembly relative to the object.

In some embodiments, the substrate connectors comprise at least one of (i) a spring clip, or (ii) hook and loop fasteners, or (iii) male and female lock elements wherein the male lock element is inserted into the female lock element and subsequently locked to the female lock element by at least one of rotation of the male and female lock elements relative to each other or lateral movement of a male lock finger relative to the female lock element, or (iv) where the second connector comprises at least one aperture extending through the substrate, or (v) an aperture through at least one of the substrate connectors in combination with a slit extending from the aperture to an outside edge of the respective substrate connector.

In some embodiments, when the disinfecting nub assembly is mounted to an object such that the object limits movement of the substrate body, a given disinfecting nub can be grasped and removed from the substrate against restraint of the object, by a worker using only one hand to so remove the nub from the substrate.

In some embodiments, the disinfectant carrier occupies substantially all of the nub cavity while providing void spaces within the disinfectant carrier for accommodating a disinfectant material therein.

In some embodiments, a disinfectant material is disposed in the disinfectant carrier, thus wetting the disinfectant carrier, and wherein the nub, when peeled away from the substrate body, can be squeezed to expel a portion of the disinfectant carrier from the nub cavity whereupon a user can use the expelled portion to scrub, and thereby to disinfect, a medical access port.

In some embodiments, a given nub extends through an aperture in the substrate, a disinfectant material is disposed in the disinfectant carrier, a sealing tab covering, and sealing, the openable end of the given nub, the sealing tab comprising a cover portion which covers and releasably seals the openable end of the given nub, and an overhang portion, extends from the cover portion, the overhang portion including a tether which is affixed to an adjacent portion of the substrate such that a user can grasp the given nub and remove the respective nub from the corresponding aperture in the substrate while peeling the openable end of the respective nub away from the sealing tab, thereby to expose the disinfectant carrier and the disinfectant material, while the tether retains the sealing tab affixed to the substrate.

In some embodiments, the housing is rigid and performs as a cap adapted to be removably mounted on a medical access port until such time as the medical access port is to be used to transfer a medical product.

In some embodiments, the sidewalls of the housing taper inwardly from the openable end toward the closed end whereby a cross-section of the nub cavity at the dosed end has a smaller area than a cross-section of the nub cavity at the openable end, the tapering of the sidewalls facilitating expelling the disinfectant carrier from the cavity by the squeezing of the nub.

In some embodiments, a disinfectant material is disposed in the disinfectant carrier, thus wetting the disinfectant carrier, and a medical access port, on an intravenous fluid tubing assembly being used on a patient, can be inserted into the openable end of the housing and the housing subsequently squeezed about a longitudinal axis of the nub, and at least one of the nub and the medical access port rotated relative to the other while maintaining the squeezing, thus to apply a tangential scrubbing force to respective surfaces of the medical access port.

In some embodiments, the housing is made of material which embodies sufficient dead fold properties that, after the squeezing is relaxed, the dead fold properties of the housing are effective to retain the medical access port in the nub through a remainder of the use of the tubing assembly on the patient.

In some embodiments, the invention comprehends a system comprising the disinfecting nub assembly mounted to an appendage of a healthcare worker.

In a second family of embodiments, the invention comprehends a disinfecting nub assembly for assisting in preventing healthcare-associated infections, the disinfecting nub assembly, and comprising a substrate, comprising a substrate body, the substrate body having opposing surfaces, and opposing first and second ends, and a substrate body length between the first and second ends; a plurality of disinfecting nubs mounted to the substrate body, a given disinfecting nub comprising a housing, the housing having an openable end and a closed end, and sidewalls extending between the openable end and the closed end, thus to define a nub cavity inside the housing, a disinfectant carrier being disposed in the nub cavity; and first and second substrate connectors associated with the opposing ends of s the aid substrate body, the substrate connectors having connector lengths extending away from the respective ends of the substrate body, the first and second substrate connectors including tethers, the tethers being adapted and configured to tether spaced locations of the disinfecting nub assembly, proximate the opposing ends of the substrate to a given object.

In some embodiments, the tethers, when the disinfecting nub assembly is tethered to a such object, being loosely mounted to the object so as to accommodate sliding movement of the tethers, and thus the disinfecting nub assembly, along a length of the object.

In some embodiments, the first connector comprises at least one of (i) a hook which engages about such object, or (ii) an aperture through at least one of the substrate connectors and a slit extending from the aperture to an outside edge of the respective substrate connector, or (iii) a spring clip, or (iv) a remote end of at least one of the first and second substrate connectors can be extended about the object and, while so extended about the object, connecting to a relatively proximal portion of the same respective substrate connector using hook and loop fasteners, thus to form a tether loop extending entirely about the respective object, or (v) at least one of the first and second substrate connectors comprises at least one aperture extending through the proximal portion of the respective tether.

In some embodiments, a disinfectant material is disposed in the disinfectant carrier, and a closure is sealed over the openable end of a given the disinfecting nub, thus to close off the openable end of the respective disinfecting nub, the closure comprising a line of weakness therein.

In some embodiments, a disinfectant material is disposed in the disinfectant carrier, thus wetting the disinfectant carrier, and the nub, when the disinfectant carrier is exposed at the openable end of the housing, can be squeezed to expel a portion of the disinfectant carrier from the nub cavity whereupon a user can use the expelled portion to scrub, and thereby to disinfect, a medical access port.

In some embodiments, the first and second substrate connectors are tethered to an intravenous tubing assembly, and wherein the disinfecting nub assembly, while so tethered to the intravenous tubing assembly, can slide freely along a length of the intravenous tubing assembly.

In a third family of embodiments the invention comprehends a disinfecting nub assembly for assisting in preventing healthcare-associated infections, the disinfecting nub assembly, having an assembly length, and comprising a substrate, comprising a substrate body, the substrate body having opposing surfaces, and opposing first and second ends, and a substrate body length between the first and second ends; a plurality of disinfecting nubs mounted to the substrate body, a given disinfecting nub comprising a housing, the housing having an openable end and a closed end, and sidewalls extending between the openable end and the closed end, thus to define a nub cavity inside the housing, a disinfectant carrier being disposed in the nub cavity, the disinfectant carrier being adapted to carry a disinfectant material therein; and a substrate extension extending from the first end of the substrate body, the substrate extension being adapted to mount the disinfecting nub assembly to an article of clothing, or a clothing appurtenance.

In some embodiments, the substrate extension is relatively more rigid than the substrate body and the substrate extension extends in a generally U-shaped configuration, thereby to define a mounting cavity for receiving an edge of the article of clothing or clothing appurtenance.

In some embodiments, when the substrate extension is mounted to such article of clothing or clothing appurtenance, the substrate body extends downwardly, drawn by gravity, from a joinder of the substrate body and the substrate extension.

In some embodiments, the substrate extension is devoid of any of the disinfecting nubs.

In some embodiments, a first portion of the substrate extension, when displaced from a second different portion of the substrate extension, is resiliently biased against the second different portion of the substrate extension and exerts a resilient force against the article of clothing or clothing appurtenance in the mounting cavity.

In some embodiments, when the disinfecting nub assembly is mounted to a user's clothing or clothing appurtenance, the mounting limits movement of the substrate body such that a given disinfecting nub can be grasped and removed from the substrate against restraint by the clothing or clothing appurtenance, the user using only one hand to so remove the nub from the substrate.

In some embodiments, the invention comprehends a disinfecting system wherein the disinfecting nub assembly is mounted to an article of clothing, or a clothing appurtenance, of a user.

In a fourth family of embodiments, the invention comprehends a disinfecting nub for assisting in preventing healthcare-associated infections in a medical environment, the disinfecting nub comprising a housing having an openable end and a closed end, and sidewalls extending between the openable end and the closed end, thus to define a nub cavity inside the housing, the housing being manually deflectable toward a longitudinal axis which extends from the openable end to the closed end, to substantially collapse the nub housing; a disinfectant carrier disposed in the nub cavity; a disinfectant material disposed in the disinfectant carrier in the nub cavity, thus wetting the disinfectant carrier; and a seal closing the openable end of the housing, wherein a medical access port, on an intravenous fluid tubing assembly being used on a patient, can be inserted into the openable end of the housing and subsequently squeezed about a longitudinal axis of the nub, and at least one of the nub and the medical access port rotated relative to the other while maintaining the squeezing, thus to simultaneously apply a tangential scrubbing force to substantially all respective surfaces of the medical access port.

In some embodiments, the housing is made of materials which embody sufficient dead fold properties that, after the squeezing is released, the dead fold properties of the housing are effective to retain the medical access port in the nub through a remainder of the use of the tubing assembly on the patient.

In some embodiments, the sidewalls have nominal taper angle, from the openable end to the closed end, of about 5 degrees to about 25 degrees, optionally about 7 degrees to about 23 degrees, optionally about 12 degrees to about 15 degrees relative to the longitudinal central axis, and wherein the taper angle is relatively constant about a circumference of said nub housing.

In some embodiments, the sidewalls of the housing have thicknesses of about 3 mils to about 45 mils, optionally about 5 mils to about 25 mils, optionally about 10 mils to about 20 mils, in facilitating the squeezing of the nub sidewalls.

In some embodiments, the housing is made from a composite material comprising a polymeric layer, a metal foil layer, and a paper layer, the metal foil layer being between the polymeric layer and the paper layer.

In a fifth family of embodiments, the invention comprehends a method of disinfecting a medical access port, comprising mounting, on a user's body, a disinfecting nub assembly comprising a substrate, including a substrate body, and opposing first and second ends, and a substrate body length between the first and second ends, a plurality of disinfecting nubs mounted to the substrate body, a given disinfecting nub comprising a housing, the housing having an openable end and a closed end, and sidewalls extending between the openable end and the closed end, thus to define a nub cavity inside the housing, a disinfectant carrier being disposed in the nub cavity, a disinfectant material being disposed in the disinfectant carrier, thus wetting the disinfectant carrier, and first and second substrate connectors associated with opposing ends of the substrate body, the first and second substrate connectors having connector lengths extending away from the respective ends of the substrate body; the mounting on the user's body comprising wrapping the first and second substrate connectors about a portion of the user's body and connecting the first and second substrate connectors to each other, thereby to mount the disinfecting nub assembly to the respective portion of the user's body; releasing a nub from the substrate and opening the respective nub at the openable end of the respective nub, thereby exposing the disinfectant carrier and the respective disinfectant material; and applying the disinfectant carrier, and thus the disinfectant material, to the medical access port, thus to disinfect the respective medical access port.

In some embodiments the method further comprises adjusting the connection of the first and second substrate connectors to each other, for example loosening or tightening the connectors relative to each other about the respective portion of the users body, thereby to facilitate utility of the disinfecting nub assembly while mounted to the respective portion of the users body.

In some embodiments, the connecting of the first and second substrate connectors to each other comprises at least one of connecting hook and loop fasteners, or connecting male and female lock elements wherein the male lock element is inserted into the female lock element and subsequently locked to the female lock element by at least one of rotation of the male and female lock elements relative to each other or by lateral movement of a male lock element member relative to the female lock element, or connecting the first substrate connector to the second connector through at least one aperture extending through the substrate, or connecting at least one of the first and second connectors to an aperture which extends through at least one of the substrate connectors and a slit extending from such aperture to an outside edge of the respective substrate connector.

In some embodiments, the method comprises mounting the disinfecting nub assembly to the users body such that the mounting limits movement of the substrate body whereby the user can grasp the nub and release the nub from the substrate using only one hand.

In some embodiments, the method further comprises, during a medical procedure, and with the disinfecting nub assembly mounted on the user's body, the user moving from a first location proximate an intravenous tubing assembly being used on a patient being treated, to a second location, displaced from the first location, proximate a supply source which provides a plurality of potentially useful medications, in medication containers and, while proximate the supply source, accessing a first disinfecting nub at the disinfecting nub assembly mounted to the users body, and opening and using the accessed first disinfecting nub to disinfect a medical access port on a such medication container at the supply source, and drawing a medication through the so-disinfected medical access port.

In some embodiments, the method further comprises the user subsequently moving from the second location proximate the supply source to the first location proximate the intravenous tubing assembly being used on the patient being treated, accessing a second different disinfecting nub at the disinfecting nub assembly mounted to the user's body, and disinfecting a second medical access port on such intravenous tubing assembly using the second different disinfecting nub.

In some embodiments, the method further comprises squeezing the nub and thereby deflecting the sidewalls of the disinfecting nub inwardly, and substantially collapsing the nub housing, causing a portion of the disinfectant carrier to protrude from the openable end of the housing, and thereafter using the protruding portion of the disinfectant carrier to scrub the medical access port.

In some embodiments, the method further comprises inserting a medical access port, on an intravenous fluid tubing assembly being used on a patient, into the openable end of a nub on the disinfecting nub assembly, subsequently squeezing the nub about a longitudinal axis of the nub, and rotating at least one of the nub and the medical access port relative to the other while maintaining the squeezing, thus applying a tangential scrubbing force to the respective surfaces of the medical access port.

In some embodiments, the method further comprises releasing the squeezing, and wherein the housing is made of material which embodies sufficient dead fold properties that, after the squeezing is released, the dead fold properties of the housing retain the medical access port in the nub cavity through a remainder of the use of the tubing assembly on that patient.

In a sixth family of embodiments, the invention comprehends a method of disinfecting a medical access port, comprising mounting, on a user's body, a disinfecting nub assembly comprising a substrate, including a substrate body, and opposing first and second ends, and a substrate body length between the first and second ends, a plurality of disinfecting nubs mounted to the substrate body, a given disinfecting nub comprising a housing, the housing having an openable end and a closed end, and sidewalls extending between the openable end and the closed end, thus to define a nub cavity inside the housing, a disinfectant carrier being disposed in the nub cavity, a disinfectant material being carried in the disinfectant carrier, a closure being sealed over the openable end of the housing, and a substrate extension extending from the first end of the substrate body, the substrate extension being adapted to mount the disinfecting nub assembly to an article of clothing, or a clothing appurtenance, the mounting on the user's body comprising mounting the adapter to an article of clothing or a clothing appurtenance on the user's body; releasing a given nub from the substrate and opening the respective nub at the openable end of the respective nub, thereby exposing the disinfectant carrier and the respective disinfectant material carried therein; and applying the disinfectant carrier, and thus the disinfectant material, to the medical access port, thus to disinfect the respective medical access port.

In some embodiments, the substrate extension is generally rigid and non-extensible such that the substrate extension accommodates releasing the nub from the substrate without substantial extension or bending of the substrate extension.

In some embodiments, the substrate extension is relatively more rigid than the substrate body and wherein the substrate extension extends in a generally U-shaped configuration, thereby to define a mounting cavity receiving such article of clothing or clothing appurtenance.

In some embodiments, a first portion of the substrate extension, when displaced from a second different portion of the substrate extension, is resiliently biased against the second portion of the substrate extension, and exerts a resilient force against the clothing or clothing appurtenance in the mounting cavity.

In a seventh family of embodiments, the invention comprehends a method of disinfecting a medical access port, comprising mounting, to an object, a disinfecting nub assembly, the disinfecting nub assembly comprising a substrate, including a substrate body, and opposing first and second ends, and a substrate body length between the first and second ends, a plurality of disinfecting nubs mounted to the substrate body, a given disinfecting nub comprising a housing, the housing having an openable end and a closed end, and sidewalls extending between the openable end and the closed end, thus to define a nub cavity inside the housing, a disinfectant carrier being disposed in the nub cavity, a disinfectant material being disposed in the disinfectant carrier, and a closure sealed over the openable end of the housing, first and second substrate connectors associated with the opposing ends of the substrate body, the substrate connectors having connector lengths extending away from the respective ends of the substrate body, the first and second substrate connectors including tethers, the tethers being adapted and configured to tether spaced locations of the disinfecting nub assembly, proximate the opposing ends of the substrate, to a given object, the mounting of the disinfecting nub assembly to the object comprising at least one of (I) mounting the first substrate connector to the object at a first location on the object and mounting the second substrate connector to the object at a second location on the object displaced from the first location, or (II) wrapping the first substrate connector about the object at a first location on the object and thereafter securing a first remote portion of the first substrate connector to a first proximal portion of the disinfecting nub assembly, and wrapping the second substrate connector about the object at a second location on the object, spaced from the first location, and thereafter securing a second remote portion of the second substrate connector to a second proximal portion of the disinfecting nub assembly; opening the respective nub at the openable end of the nub, thereby exposing the disinfectant carrier and the respective disinfectant material; and applying the disinfectant carrier, and thus the disinfectant material, to a such medical access port, thus to disinfect the respective medical access port.

In some embodiments, the object comprises an intravenous tubing assembly, the mounting comprising wrapping the first substrate connector about the intravenous tubing assembly, and thereafter securing a remote portion of the first substrate connector to a first proximal portion of the disinfecting nub assembly, and wrapping the second substrate connector about the same intravenous tubing assembly and thereafter securing a remote portion of the second substrate connector to a second proximal portion of the disinfecting nub assembly.

In some embodiments, the method comprises wrapping the first and second substrate connectors loosely about the intravenous tubing assembly such that the disinfecting nub assembly can slide freely along at least a portion of a length of the intravenous tubing assembly.

In some embodiments, the method comprises positioning the disinfecting nub assembly along the length of the intravenous tubing assembly such that a medical access port on the intravenous tubing assembly is between the first and second substrate connectors.

In some embodiments, the method further comprises orienting the disinfecting nub assembly such that the sealed ends of the disinfecting nubs face generally toward the medical access port on the intravenous tubing assembly.

In some embodiments, closures are sealed over the openable ends of respective ones of the disinfecting nubs, and such closures comprise a line of weakness, the lines of weakness optionally being disposed away from end edges of the sidewalls of the respective nub housings, the method comprising grasping a such disinfecting nub and breaching the line of weakness, thereby exposing the disinfectant carrier, and applying the disinfectant carrier, bearing the disinfectant material, to the medical access port to thereby disinfect the respective medical access port.

In some embodiments, the method includes using the medical access port to breach the line of weakness on the closure of a respective disinfecting nub.

In some embodiments, the method includes, after using the medical access port to breach the line of weakness, squeezing the nub sidewalls inwardly and thereby expelling a portion of the disinfectant carrier from the openable end of the housing, and subsequently using the so expelled portion of the disinfectant carrier, and the carried disinfectant material carried therein, to scrub the respective medical access port.

In some embodiments, the method comprises using the medical access port to breach the line of weakness on a given disinfecting nub, subsequently squeezing the nub sidewalls inwardly and thereby expelling a portion of the disinfectant carrier from the openable end of the housing, and subsequently using the so expelled portion of the disinfectant carrier, and the carried disinfectant material, to scrub, and thereby disinfect, the medical access port on the intravenous tubing assembly, all while the respective disinfecting nub remains attached to the substrate and the disinfecting nub assembly remains mounted to the intravenous tubing assembly.

In some embodiments, the given disinfecting nub comprises a first disinfecting nub, the method further comprising breaching the line of weakness on a second such disinfecting nub which is mounted on the substrate, subsequently squeezing the sidewalls on the second disinfecting nub inwardly and thereby expelling a second portion of disinfectant carrier, including the disinfectant material carried therein, from the openable end of the second nub housing, and subsequently using the so expelled second portion of disinfectant carrier from the second disinfecting nub to scrub, and thereby disinfect, the same medical access port on the intravenous tubing assembly a second time, all while the second disinfecting nub remains attached to the substrate and the disinfecting nub assembly remains mounted to the same intravenous tubing.

In some embodiments, the invention comprehends a method of disinfecting a medical access port, comprising accessing a disinfecting nub, the disinfecting nub comprising a housing having a closed end and an openable end, and sidewalls extending between the openable end and the closed end, thus to define a nub cavity inside the housing, the housing being manually deflectable toward a longitudinal axis which extends from the openable end to the closed end, to thereby substantially collapse the nub housing, a disinfectant carrier disposed in the nub cavity, a disinfectant material disposed in the disinfectant carrier in the nub cavity, and a closure sealed over the openable end of the housing; inserting a medical access port, on an intravenous fluid tubing assembly being used on a patient, into the openable end of a such nub on the disinfecting nub assembly; and subsequently squeezing the nub about the longitudinal axis of the nub and rotating at least one of the nub and the medical access port about the other while maintaining the squeezing, thus applying a tangential scrubbing force to the respective surfaces of the respective medical access port.

In some embodiments, the method further comprises releasing the squeezing, and the housing is made of material which embodies sufficient dead fold properties that, after such squeezing, the dead fold properties of the housing retain the medical access port in the nub through a remainder of the use of the tubing assembly on such patient.

In some embodiments, the closure sealed over the openable end of the housing comprises a line of weakness, the method comprising using the medical access port to breach the line of weakness.

In some embodiments, the method further comprising, after squeezing the nub and thereby deflecting the sidewalls of the disinfecting nub inwardly and causing the portion of the disinfectant carrier to protrude from the openable end of the housing, at least partially releasing the squeezing of the nub sidewalls and inserting the medical access port into the openable end of the housing, thereby mounting the disinfecting nub onto the medical access port.

### BRIEF DESCRIPTION OF THE DRAWINGS

FIG. 1 shows a plan view of an operating room, with an operation in progress.

FIG. 2 is an pictorial view of a medication vial commonly used to store and distribute liquid medications.

FIG. 3 shows an elevation view of a portion of an intravenous fluid tubing assembly commonly used to administer liquid medications to a patient.

FIG. 4 is a pictorial view of a first embodiment of nub assemblies of the invention, including a first embodiment of nub substrates of the invention.

FIG. 5 is a pictorial view of a second embodiment of nub substrates of the invention, including a male/female lock combination on a substrate connector, where the male and/or the female lock elements twist/rotate in locking the male and female lock elements to each other.

FIG. 6 is a pictorial view of a second embodiment of nub assemblies of the invention, using the nub substrate of FIG. 5.

FIG. 7 shows a cross-section of a first embodiment of a disinfecting nub of the invention mounted on a substrate, and taken at 7-7 of FIG. 6.

FIG. 8 shows a plan view of a nub assembly of the invention, including a male/female lock combination on the substrate connector where the male lock fingers move laterally in locking to the female lock element.

FIG. 9 shows a pictorial view of a nub assembly mounted to a user's arm, and the user accessing one of the nubs for release/removal of the nub from the substrate body.

FIG. 10 is a side elevation view of a nub assembly which includes a substrate adapter having a generally U-shaped upper end which attaches to a user's belt or other edge of the user's clothing or clothing appurtenance.

FIG. 11 is a front elevation view of a user wearing a belt, and the nub assembly of FIG. 10 mounted to the belt.

FIG. 12 shows a cross-section of a second embodiment of a disinfecting nub of the invention, with the openable end of the nub mounted to a substrate.

FIG. 13 shows a cross-section of a nub as in FIG. 12 including an invagination/cut in the disinfectant carrier, the cut extending from the openable end of the nub toward the closed end of the nub.

FIG. 14 shows a cross-section of a nub as in FIG. 13 including a recess in the disinfectant carrier in place of the invagination/cut.

FIG. 15 shows a cross-section of a nub similar to the nub of FIG. 13, but wherein the closed end of the nub is flat such that the closed end of the nub can be mounted to the substrate, and a sealing tab over the openable end of the substrate can be displaced, opened by squeezing the sidewalls of the nub.

FIG. 16 is a cross-section of a nub where the seal over the openable end of the nub has been removed and the nub sidewalls have been squeezed to expel a portion of the disinfectant carrier to create a protruding surface for scrubbing a medical access port.

FIG. 17 is a cross section of a nub as in FIG. 16 after the nub has been allowed to relax after scrubbing the medical access port, and the port has then been inserted into the disinfectant carrier at the cut/invagination.

FIG. 18 shows a nub of the invention wherein the medical access port has been inserted into the cut/invagination and the nub has subsequently been squeezed to create dead folds in the nub housing.

FIG. 19 shows the nub of FIG. 18 after the squeezing force has been withdrawn, showing that the dead folds are effective to hold the previously-squeezed nub on the port.

FIG. 20 is a pictorial view of yet another embodiment of a nub assembly of the invention wherein each of the substrate connectors is connected to tubing of an intravenous fluid tubing assembly on opposing sides of a medical access port, and wherein the substrate connectors mount to the tubing through slits which extend from apertures in the substrate connectors to remote edges of the substrate.

FIG. 20A shows another embodiment of a nub assembly of the invention wherein each of the substrate connectors connects individually to tubing of an intravenous fluid tubing assembly wherein each substrate connector forms a loop extending entirely about the tubing.

FIG. 21 is a pictorial view showing lines of weakness in the seal tab which is sealing the openable end of the nub, and cuts, invaginations in the disinfectant carrier being shown as dashed lines.

FIG. 22 is a pictorial view of the nub of FIG. 21 after the lines of weakness have been breached and the nub sidewalls have subsequently been squeezed, expelling a portion of the disinfectant carrier.

FIG. 23 is a cross-section of one embodiment of material which can be used for the sidewalls of the nub.

The invention is not limited in its application to the details of construction, or in the arrangement of the components, or in the specific methods set forth in the following description or illustrated in the drawings. The invention is capable of other embodiments or of being practiced or carried out in various other ways. Also, it is to be understood that the terminology and phraseology employed herein is for purpose of description and illustration and should not be regarded as limiting. Like reference numerals are used to indicate like components.

### DETAILED DESCRIPTION OF THE ILLUSTRATED EMBODIMENTS

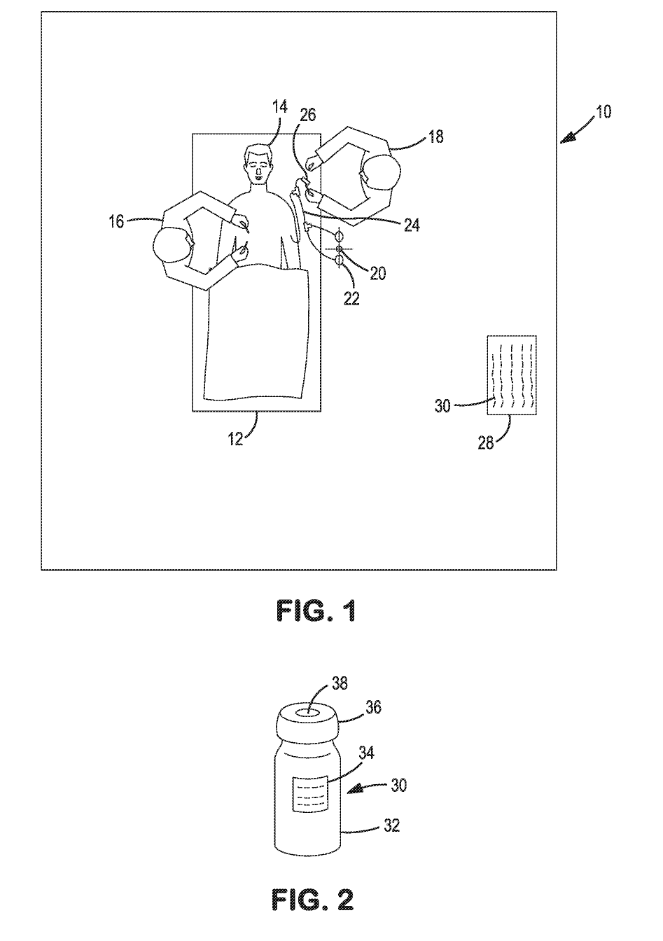

Referring to FIG. 1, in an operating room 10, an operating table 12 is used to support a patient 14 while a surgeon 16 and/or other medical professionals render medical services to the patient. As part of the medical services rendered to the patient, an anesthesia professional 18 administers various fluid medications to the patient. An intravenous fluid stand 20 supports one or more bags 22 of intravenous liquids. Intravenous fluids in fluid bags 22 are fed to an intravenous fluid tubing assembly 24. The intravenous fluid tubing assembly is connected to a catheter which has been inserted into a vein in the patient.

Fluids from the bags 22 are commonly fed drop-wise into tubing assembly 24, and by gravity through the tubing assembly into the vein of the patient, and thus into the patient's circulating blood stream. Additional fluids, including medications, can be added to the fluid composition in tubing assembly 24 through one or more medical access ports 26 in the tubing assembly. Containers of such fluids and/or medications are commonly available to the anesthesia professional on a medications cart 28 in the operating room, but which cart is typically located some distance from the operating table, out of reach of the anesthesia professional.

FIG. 2 shows a typical medication vial 30, as an example of containers used to contain medications on the medications cart. Vial 30 includes a vial body 32, typically glass or plastic, which serves as a receptacle to hold a fluid medication. A label 34 states the identity of the medication contained in vial 30. A vial cap 36, typically metal, serves as a closure providing a seal to prevent ingress of foreign material into, or unintended egress of the medication from, the vial. Cap 36 has a central opening which is closed by a pierceable, typically rubber, septum 38. Septum 38 can be pierced by a needle, e.g. on a syringe, for the purpose of withdrawing a desired quantity of the liquid medication, contained in vial 30, into the syringe receptacle without exposing, to ambient air, either the medication drawn into the syringe or the medication which remains in the vial after a portion of the medication has been withdrawn.

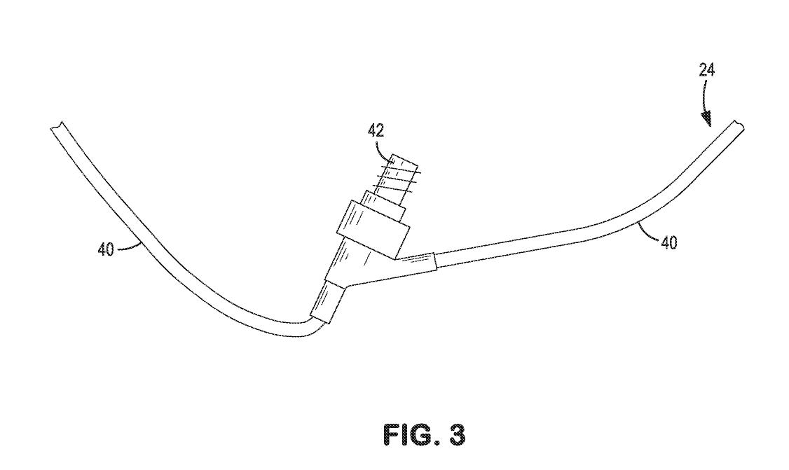

FIG. 3 illustrates a portion of intravenous tubing assembly 24, including first and second lengths of tubing 40 connected to a medical access port, specifically to a luer connector 42 being illustrated as the medical access port.

Any time the anesthesia professional, or any other healthcare professional, needs to access a medication, or to administer a medication through e.g. the intravenous fluid tubing assembly, or to otherwise transfer a medication from a first container to a second container or to an intravenous tubing assembly, all surfaces associated with the medication transfer need to be disinfected. Any needle/syringe to be used for such transfer must be disinfected. Where the syringe is a single-use item, the syringe is typically disinfected, and maintained disinfected, before and while being packaged at the manufacturer or other distributing source for the syringe, whereby the syringe need not be disinfected if used immediately after being removed from its packaging.

Before a medication is withdrawn through a septum, using such single-use syringe bearing a needle, the septum must be disinfected. Before a medication is introduced into the intravenous fluid tubing assembly at a medical access port such as at luer connector 42, the port needs to be disinfected.

FIG. 1 illustrates that the intravenous fluid tubing assembly and the medication cart are at two different locations, such that the anesthesia professional moves back and forth between the two different locations in order to access needed medications as well as to administer such medications through the intravenous fluid tubing assembly. Given that both the medication containers on the cart and the access port/luer connector on the tubing assembly need to be disinfected for each medication used, the anesthesia professional needs to have disinfectant materials available at both the intravenous fluid tubing assembly and at the medication cart.

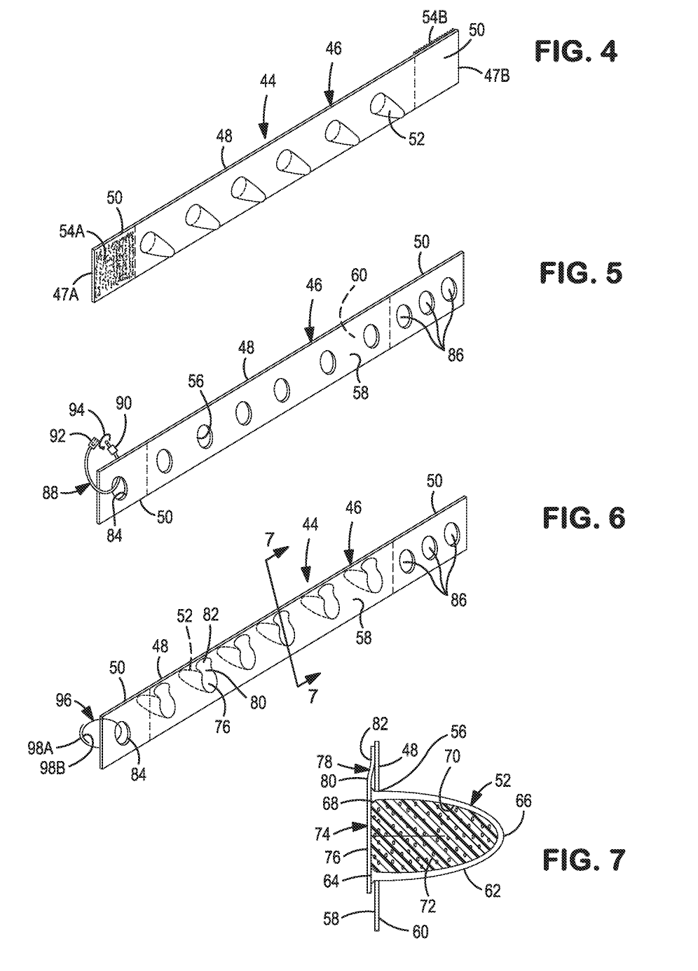

FIGS. 4-6 and 8 illustrate nub assemblies 44 which are adapted and configured to be mounted to a user's body such that a respective such nub assembly moves with the anesthesia professional or other healthcare professional as the user moves back and forth between and among the multiple work stations.

Referring now to FIG. 4, nub assembly 44 includes a substrate 46 which extends from a first end 47A to a second end 47B of the nub assembly. Substrate 46 includes a central substrate body 48 and substrate connectors 50 on opposing ends of the substrate body. A plurality of disinfecting nubs 52 are mounted to the substrate body. The substrate connectors 50 are devoid of nubs 52.

In the embodiments illustrated in FIG. 4, hook and loop fasteners 54A, 54B are mounted on opposing surfaces of the substrate connectors such that the substrate can be wrapped about an object and mounted to that object using the hook and loop fasteners to make the connection between the opposing substrate connectors.

Referring now to FIGS. 5-7, six nub apertures 56 are spaced along the length of substrate body 48, extending from a first surface 58 of the substrate body, facing the viewer, to an opposing second surface 60 of the substrate body, facing away from the viewer. In FIG. 6, six disinfecting nubs 52 extend through the respective six apertures 56.

Referring to FIGS. 6 and 7, a nub 52 has a nub housing 62. Housing 62 has an openable end 64 and a closed end 66. In the embodiments illustrated in FIGS. 6 and 7, housing 62, and thus nub 52, extends through the respective aperture 56. Openable end 64 of the nub housing is in front of first surface 58 of the substrate body, facing toward the viewer. As illustrated in FIG. 7, housing 62 may be configured with a flange 68 having a diameter greater than the diameter of the respective aperture 56. Flange 68 serves as a temporary stop, or locator, assisting with locating the openable end of the housing on the first-surface side of the substrate body during assembly of nub assembly 44. Accordingly, the openable end of the housing extends slightly frontwardly, toward the viewer, from the first surface of the substrate body. The housing extends, from the openable end of the housing, rearwardly through the respective aperture 56, with the closed end of the housing being disposed substantially rearwardly of the second surface of the substrate body, away from the viewer.

As illustrated in FIG. 7, the inside of the housing defines a cavity 70. A disinfectant carrier 72, for carrying a disinfecting material, is disposed in, and substantially fills, the cavity. Disinfectant carrier 72 thus extends from closed end 66 of the housing to a location very close to openable end 64 of the housing. Thus, the disinfectant carrier substantially fills the cavity inside the nub housing.

Where the disinfecting material is a liquid, typically a saturating load of the disinfecting liquid is disposed in the disinfecting carrier.

As the disinfecting material there can be mentioned, for example and without limitation, such liquids as isopropyl alcohol at known medical concentrations, povidone iodine, chlorhexidine gluconate, and hydrogen peroxide. In some instances where a dry disinfectant may be desired, there can be mentioned as the disinfecting material certain of the metals in powder form, such as gold, zinc, copper, cerium, and silver.

In the embodiments illustrated in FIGS. 5-7 each nub 52 has a separate sealing tab 74. A sealing tab 74 has a cover portion 76 which sealingly covers the openable end of the nub. The sealing tab also includes an overhang portion 78. The overhang portion includes a tether 80 of sufficient length that a tip 82 of the tether overlaps an adjacent portion of the substrate body and is affixed to the substrate body so that the corresponding nub is tethered to the substrate body by sealing tab 74 at tip 82.

While flanges 68 are illustrated and discussed, and cover portion 76 is sealed to flanges 68, flanges 68 are optional so long as the material used for sealing tab 74 can sealingly close the openable end of the housing.

As illustrated in FIG. 7, the remote ends of flanges 68 are not co-planar with first surface 58 of the substrate body. Rather, tether 80 serves as a bridge between cover portion 76 and sealing tip 82.

To remove a nub from the nub assembly, the user grasps the nub housing on the side of the substrate body which is seen facing away from the viewer in FIG. 6. The user then pulls on the nub housing, pulling the openable end of the housing, including flange 68, through the respective aperture 56 and away from the substrate. As the user pulls the nub housing away from the substrate, tip 82 of the tether retains the sealing tab mounted on the substrate at first surface 58 while cover portion 76 of seal tab 74 peels away from the openable end of the housing. Thus, the process of removing the nub from the nub assembly at substrate body 48 also removes the seal tab from the openable end of the housing, whereby disinfectant carrier 72, and the liquid carried in the disinfectant carrier, become exposed and available for immediate use.

Returning to FIGS. 5 and 6, as with FIG. 4, substrate connectors 50 are configured for connecting the nub assembly to an object, such as to a portion of a user's body. In FIGS. 5 and 6, the substrate connector at end 47A of the substrate has a single aperture 84. The substrate connector at end 47B of the substrate has three apertures 86.

In FIG. 5, a flexible ring 88 extends through the single aperture 84 in the connector proximate end 47A. A male end 90 of ring 88 can be inserted into a female end 92 of ring 88. The male and female ends of ring 88 are configured to lock together by a rotational, twisting motion relative to each other, as indicated by the arcuate arrow 94. Locking rings 88, using the twisting lock motion, are available, labeled as key rings for holding an assortment of keys.

With nubs mounted to the nub substrate, ring 88 is inserted through aperture 84 as shown in FIG. 5. The nub assembly is then wrapped about an object, such as a user's arm, or a pouch, or a belt, or other item of the user's clothing or clothing appurtenance. The ring in aperture 84 is then extended through one of the apertures 86 on the opposing substrate connector and the male and female ends are connected to each other with the above-mentioned twist locking motion, thus making the connection between the opposing substrate connectors, which completes the step of mounting the nub assembly to the user/object. If the nub assembly is then too tight or too loose relative to the object to which the nub assembly is mounted, the lock is released, and the tightness, looseness is adjusted by selecting a different one of apertures 86, and the lock is again locked, with the newly-selected aperture 86 so engaged by the lock.

FIG. 6 shows the same substrate as FIG. 5, but with nubs mounted in the apertures 56, and with a different type of lock mechanism, namely a spring clip 96 in the form of a ring. Spring clip 96 is made of spring steel, or similar springing material which can be deformed, deflected, and will resiliently return to its previous shape, for example a split-ring key ring. The overlapping ends 98A, 98B can thus be temporarily deformed so the ring can be mounted to aperture 84 as illustrated in FIG. 6. As with the embodiment illustrated in FIG. 5, the nub assembly is then wrapped about an object, such as a user's arm, or a pouch, or a belt, or other item of the user's clothing or clothing appurtenance. The ends 98A, 98B are then deformed enough that one of the ends can be extended through one of the apertures 86 on the opposing substrate connector, thereby mounting ring on the respective aperture 86 as well as on aperture 84, thus making the connection between the opposing substrate connectors, which completes the step of mounting the nub assembly to the object/user. If the nub assembly is then too tight or too loose relative to the object to which the nub assembly is mounted, the lock is released, and the tightness, looseness is adjusted by selecting a different one of apertures 86, and the lock is again locked, with the newly-selected aperture 86 so engaged by the lock.

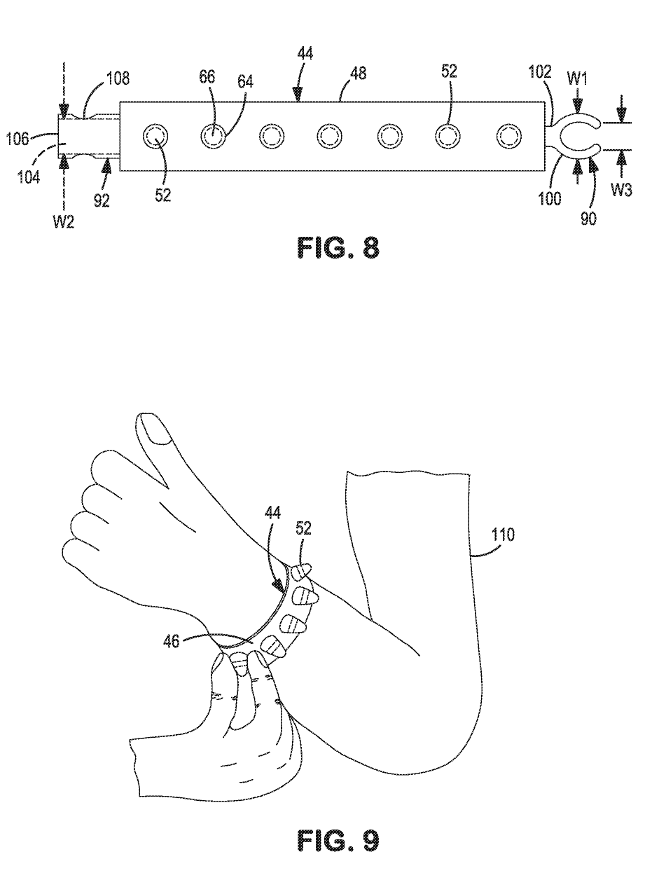

FIG. 8 shows a substrate body where, as in FIG. 4, nubs 52 are mounted to the substrate body at openable ends 64. Substrate connectors 50 are illustrated in the form of male and female lock elements 90 and 92. Male lock element 90 has first and second resiliently deflectable lock fingers 100 extending from a base 102 which is attached to the substrate body. Female lock element 92 is attached to the opposing end of the substrate body, from the male lock element. Female lock element 92 has an opening 104 which extends, from a remote end 106 of the female lock element, to a location adjacent the substrate body. A pair of opposing side apertures 108 are disposed on the sides of the female lock element intermediate the remote end of the female lock element and the proximal end of the female lock element.

The greatest dimension “W1” between the outer edges of lock fingers 100 is greater than the greatest width “W2” at the inner surfaces of opening 104 along the length of the opening. The width dimension “W3” between the remote ends of lock fingers 100 is less than the greatest width “W2” in the female lock element.

The nub assembly 44 of FIG. 8 is mounted to an object by wrapping the nub assembly, including the substrate body, about the object to be mounted to. With the nub assembly wrapped about the object, resiliently deflectable lock fingers 100 on male lock element 90 are inserted into opening 104 at the remote end of female lock element 92. Lock fingers 100 deflect resiliently toward each other as the lock fingers enter opening 104. As the greatest width portions of the male lock fingers reach apertures 106, fingers 100 resiliently extend away from each other into apertures 106, thus locking the male and female lock elements to each other and securely mounting the nub assembly to the object.

While a number of embodiments of substrate connectors have been illustrated, such are only exemplary of connection structures which can be used. Any other known connecting mechanisms may be used as well. Where the nub assembly is intended to be mounted about a user's arm, substrate connectors are typically selected for ease of mounting using a single hand. To that end, the hook and loop fastener connectors are an excellent choice because of the ease of attaching the hook end and the loop end to each other using a single hand. In addition to being easy to attach to each other, the hook and loop fasteners lend themselves well to length adjustments during the attachment process, or to readjusting length where the initial mounting is looser or tighter than desired.

Length adjustability is desirable in order to be able to attach the nub assembly to a variety of sizes of objects, for example the arms/wrists of users having relatively larger or smaller arm/wrist sizes. For example, where hook and loop fasteners are used as the attachment mechanism of choice, either or both of the substrate connectors may be designed, fabricated longer than shown in FIG. 4.

Another method of making the length of the nub assembly adjustable is to use resiliently extensible/elastic material for either or both of substrate body 48 or the substrate elements embodied in substrate connectors 50. However, where nubs 52 are releasably sealed to the substrate body at openable ends 64 as in FIG. 4, substrate body 48 cannot be so extensible as to prematurely breach the nub-to-substrate seals when the substrate is extended during mounting of the nub assembly to a target mounting object. Accordingly, while substrate connectors 50 can have substantial extensibility, e.g. up to about 100 percent elongation to an effective stop upon manual pulling, the extensibility of the substrate body is typically limited to less than 20 percent elongation, optionally less than 10 percent elongation, to an effective stop upon manual pulling. By manual pulling is meant the force which can be readily exerted by an average adult when mounting the nub assembly about that person's arm using a single hand.

FIG. 9 shows a nub assembly of the invention so mounted to an arm 110 of a user. While the substrate connectors are not shown in FIG. 9, the hook and loop fasteners are the connectors of choice because of the ease of attaching the fasteners on the substrate connectors to each other using a single hand.

FIG. 9 further illustrates that the nub assembly, mounted as a band about the user's arm/wrist, is snug against the arm/wrist of the wearer such that the band resists any force pulling the band laterally away from the arm. Thus, when the user desires to use a nub 52, the user simply grasps a selected nub as shown in FIG. 9 and pulls the nub away from the band. As the nub is pulled away from the band, the band/substrate resists such pulling while the pulling force peels the nub away from the substrate body, exposing the openable end of the nub, as well as the contained disinfectant carrier and the disinfecting liquid carried by the disinfectant carrier.

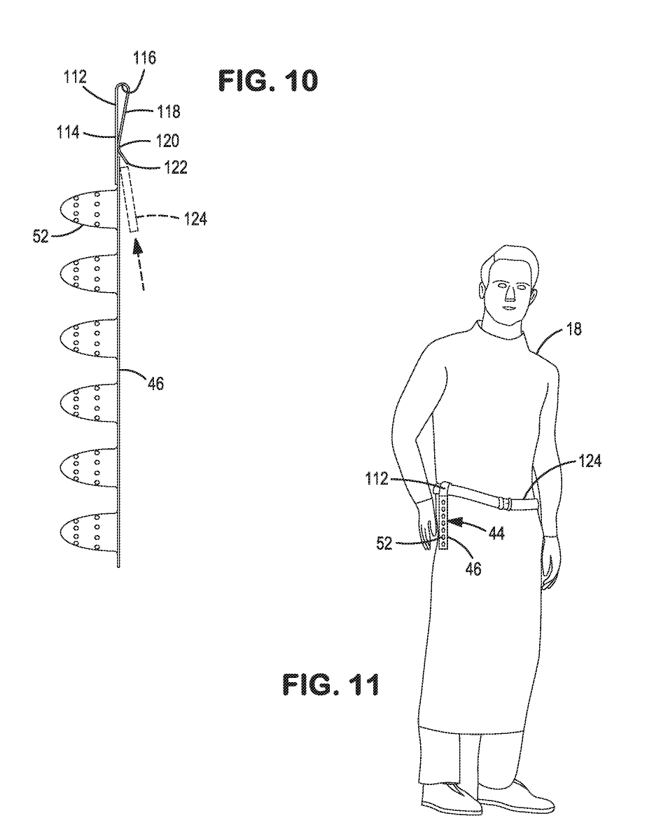

FIGS. 10 and 11 show yet another embodiment of nub assemblies 44 of the invention. Openable ends 64 of nubs 52 are releasably sealed to first surface 58 of substrate body 48. The nub assembly of FIGS. 10 and 11 has no substrate connectors per se. Rather, a substrate adapter 112 is secured to first surface 58, or optionally second surface 60, of the substrate at end 47A of the substrate. While substrate 46 is typically a flexible e.g. plastic material, substrate adapter 112 is a generally more rigid member. As illustrated, but not as a requirement, substrate adapter 112 extends from end 47A in a direction generally consistent with the length of the substrate to which the substrate adapter is connected. As illustrated, and consistent with orientation of the nub assembly, substrate adapter 112 extends upwardly as a first leg 114 to a bight 116, about the bight and thence downwardly as a second leg 118 and meeting first leg 114 at a location 120 below the bight and above the locus of attachment of the first leg to the substrate. From location 120, the second leg of adapter 112 is flared outwardly away from first leg 114 as shown, to a distal end 122. FIG. 10 illustrates that the outward flare at distal end 122 is far enough from first leg 114 that the flared portion of the adapter can receive the thickness of a user's belt 124, shown in dashed outline in FIG. 10, between the end of the flare and the first leg. Thus, the substrate adapter can be used to mount the nub assembly to the user's belt. As the substrate adapter is e.g. pushed down on the belt, moving the belt up toward bight 116, the belt thickness deflects second leg 118 resiliently away from first leg 114, such that the restorative forces developed between first and second legs 114, 118 develop substantial frictional engagement between the belt and the substrate adapter, thus firmly holding the nub assembly mounted on the belt.

In the alternative, the flare on the substrate adapter can be similarly used to mount the substrate adapter, and thus the nub assembly, to a strap, or a fold, or a handle of a pouch, or other appurtenance of the user's clothing, or to a side rail of the medication cart, or other object where the nub assembly is expected to be useful. Thus, as with the substrate connectors, the substrate adapter provides a versatile tool for mounting the nub assembly to a wide variety of objects.

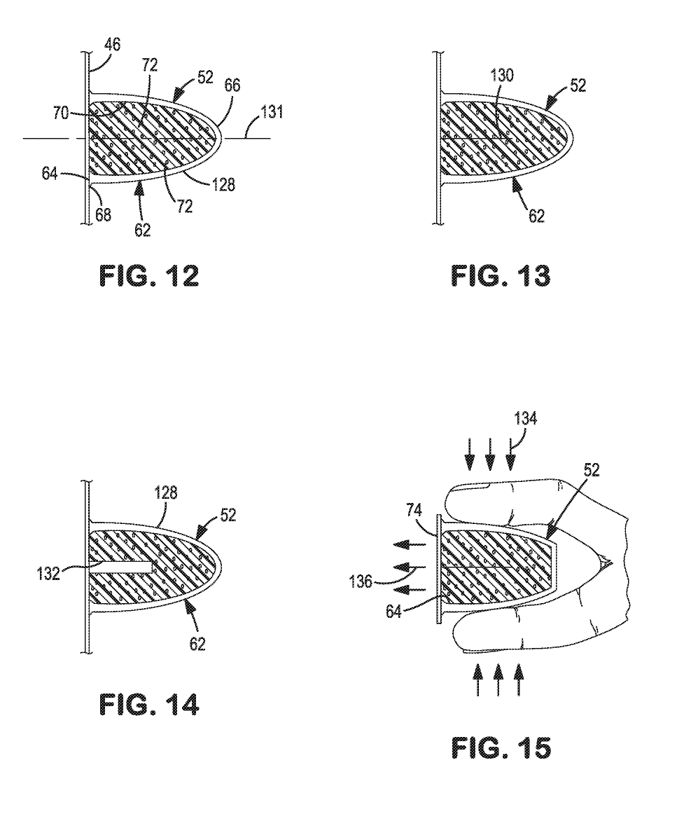

FIG. 12 shows a cross-section of a nub of the invention wherein substrate 46 is mounted and sealed over openable end 64 of the housing at flanges 68. As illustrated in FIG. 12 housing 62 is generally tapered from the openable end of the housing, inwardly toward the closed end of the housing. The taper can extend in a straight line, or can be generally arcuate as shown in the drawings. The actual path traversed by housing sidewalls 128 between the openable end and the dosed end is not critical; however a general taper is beneficial for reasons described following.

Substrate 46, or in some instances a separate seal tab 74, can be sealed to the nub housing using, for example and without limitation, heat sealing, other thermal bonding, adhesive sealing such as with silicone, silicone rubber, methyl methacrylate, or the like, sonic bonding microwave welding, or induction heating, among others.

Disinfectant carrier 72 generally fills cavity 70 inside housing 62, from closed end 66 to overlying material to which the openable end is sealed. Material for the disinfectant carrier can be selected from any known absorbent material which is acceptable for use in medical implementations. Exemplary such disinfectant carrier materials are, for example and without limitation, non-wovens and foams, particularly open-cell foams, namely liquid-permeable foams, typically foams which are resiliently-deformable and/or otherwise elastic.

In the dosed and sealed state, the openable end is closed and sealed by a covering substrate or tab. In that closed and sealed state, disinfectant carrier 72 may be in a slightly compressed condition such that when the seal is breached at a tab 74 (FIGS. 7 and 15) or substrate 46 (FIG. 12), the disinfectant carrier may automatically protrude a short distance beyond the openable end of the housing, by recovery from that slight compression.

FIG. 13 shows a cross-section of a nub of the invention, generally as in FIG. 12. However, the nub of FIG. 13 includes a cut, invagination 130 extending, from the surface of the disinfectant material carrier which is exposed proximate openable end 64 of the housing when the seal is breached, toward the closed end of the housing. Cut, invagination 130 extends into the disinfectant material carrier a distance sufficient to receive the working end of any medical access port which is expected to be used to be cleaned, disinfected using such nub. The profile of the cut/invagination may be a simple linear slit, or one or more crossing slits or other cut configuration, typically meeting at the longitudinal axis 131 of the nub.

FIG. 14 shows a cross-section of a nub of the invention, generally as in FIGS. 12 and 13. However, the nub of FIG. 14 includes a recess 132, representing material which has been removed from the openable end portion of the disinfectant carrier. Recess 132 provides a starting location for receiving a medical access port thereinto by further deflection of the carrier material when the end of a medical access port is pressed into the carrier material.

FIG. 15 shows a cross-section of yet another nub of the invention. In FIG. 15, the openable end of the housing has been closed and sealed using an individual seal tab 74 which overlies only a single nub, rather than using part of the substrate as the sealing closure as shown in FIGS. 12-14.

In the embodiment of FIG. 15, closed end 66 of the housing is flat rather than rounded as in FIGS. 12-14 whereby nub 52 is mounted to the substrate at closed end 66.

To open the nub and expose the disinfectant carrier in the embodiment of FIG. 15, the user has two choices. First, the user can simply squeeze the sidewalls of the housing as suggested by arrows 134 in FIG. 15, using a single hand to open the nub. When the sidewalls of the housing are squeezed, pneumatic and/or hydraulic pressure created inside the housing by that squeezing applies sufficient force on tab 74 that the seal between the tab and flange 68 is breached as suggested by arrows 136.

As a second option, the user can use his/her second hand to grasp an edge of peel tab 74 and thereby peel the seal tab away from the openable end of the housing, again exposing the disinfectant carrier.

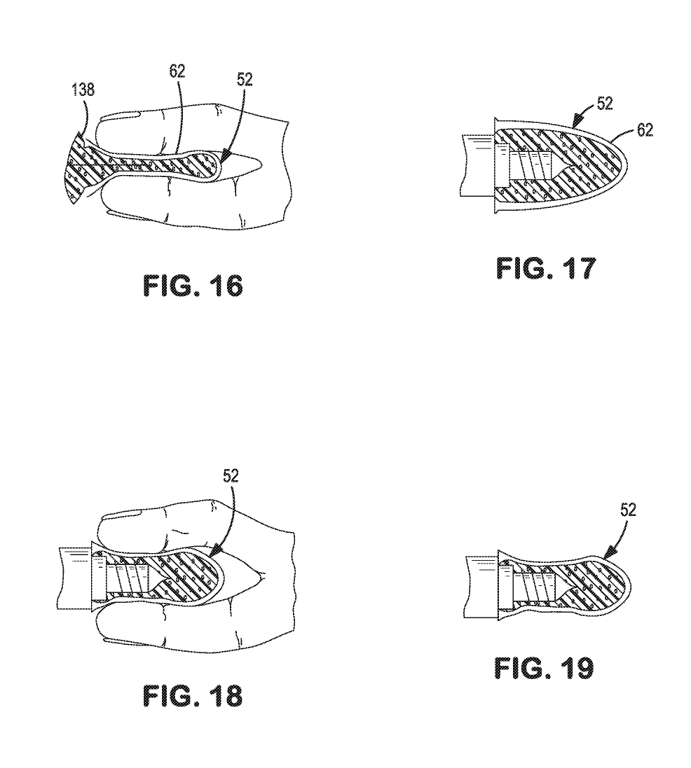

FIGS. 16 and 17 illustrate a nub which uses a resilient housing 62. As illustrated in FIG. 16, squeezing the nub housing at the tapering sidewalls results in a portion 138 of the disinfectant carrier being expelled from the housing. With the disinfectant carrier thus extending beyond the openable end of the housing, the extending portion 138 of the carrier becomes available as a cleansing surface, loaded with disinfecting material, for wiping, scrubbing, and otherwise cleansing and disinfecting exposed surfaces of a medical access port. Expelled portion 138 can be used for wiping and/or scrubbing any portion of any medical access port. The expelled portion has particular advantage for wiping the septum of a medication vial 30 (FIG. 2).

FIG. 17 shows that, once the cleansing and disinfecting process has been completed to the satisfaction of the user, the squeezing force on the housing can be released which results in the resilient housing and the resilient disinfectant carrier expanding, reverting to generally their pre-squeeze configurations. Once the housing and carrier have so expanded, the now-cleansed port, for example a luer connector illustrated in FIG. 3 which has been wiped/scrubbed using the expelled portion of the disinfectant carrier, can be inserted into the cut, invagination in the carrier, and retained in the carrier/nub, in a maintained disinfected condition, until such time as the port is next to be used.

FIGS. 18 and 19 show yet another method of cleansing, disinfecting a medical access port, and maintaining the port in a cleansed, disinfected condition. In FIGS. 18 and 19, housing 62 is made using material, typically a composite, multiple layer material, which has a good dead fold quality. A good dead fold quality can be obtained using metal foils such as aluminum foil, and some paper products. An exemplary, but not limiting, such 3-layer material which has good dead fold properties is illustrated generally in cross-section in FIG. 23. As illustrated in FIG. 23, a metal foil layer 142, such as aluminum foil, is positioned between an outer paper layer 144 and an inner e.g. heat sealable layer 146 such as polyethylene or polypropylene. Adhesive layers, not shown, can be used on opposing sides of the metal foil layer. Those skilled in the composite materials arts can determine suitable materials and materials thicknesses for the respective layers, as well as the specific e.g. paper product and polymer product to be used for the sealable layer, and any needed adhesive/bonding layers.

Returning to FIGS. 18 and 19, with the openable end of the housing open, thus with the disinfectant carrier exposed, the medical access port to be cleansed, disinfected is pushed into the disinfectant carrier. Where a cut/invagination 130, or recess 132 is available, such cut or recess facilitates the entry of the medical access port into the disinfectant carrier. The user then squeezes, crushes the sidewalls of the nub housing as suggested in FIG. 18, thusly applying forces directed generally perpendicular to respective ones of the exposed outer surfaces of the medical access port. While maintaining the squeezing force, the user rotates the nub about the medical access port. The combination squeezing and rotating provides a physical cleansing/scrubbing action, which can remove, loosen particulate material from the exposed outer surfaces of the port. The disinfectant material carried in the disinfectant carrier can then chemically disinfect the removed particulate material as well as any other loose infective material which may be present on the surface of the port.

In addition to facilitating the scrubbing process, the squeezing activates the dead fold properties of the housing materials. Once the scrubbing action has been completed, the user can then withdraw the squeezing forces from the housing and the dead fold properties of the nub housing materials continue to hold the nub housing crushed about the medical access port as illustrated in FIG. 19. With the nub held on the medical access port by the crushed housing sidewalls, the medical access port remains disinfected until such time as the disinfecting nub is withdrawn, for example for the next use of respective port. For example, when the port is to be used again, the nub is withdrawn from the port and the port is at that time still disinfected and ready for use.

While FIGS. 18 and 19 illustrate crushing the nub having dead fold properties about the port and scrubbing the port using the crushed nub, the crushable nub can also be crushed without first inserting the port into the nub, resulting in the portion 138 of the disinfectant carrier being expelled from the nub as in FIG. 16, whereupon the nub having the dead fold properties and the expelled portion of the disinfectant carrier can be used to scrub either a medication vial or a medical access port. The invention also contemplates that the crushable nub, once crushed to expel portion 138, can be used to scrub, disinfect the patient injection site.

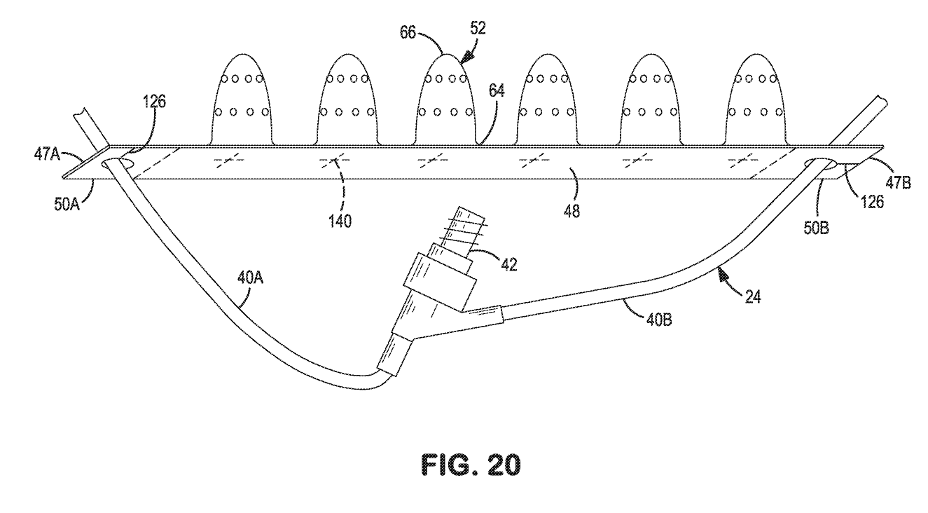

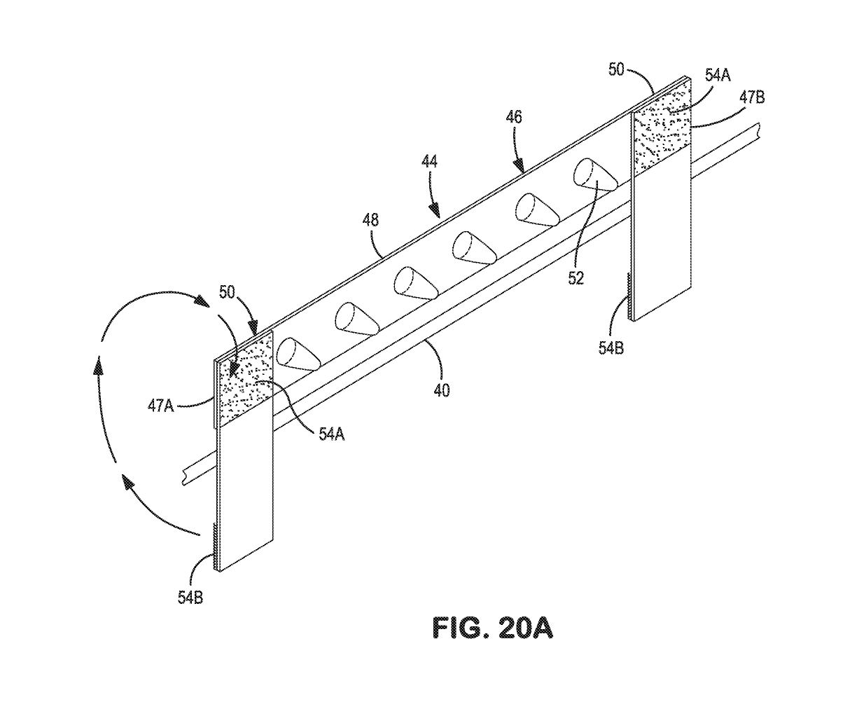

FIGS. 20, 20A, 21, and 22 show additional novel structures for the nub assembly, and novel methods of using the nub assembly to disinfect a medical access port such as a luer connector. In FIG. 20, opposing ends of a nub assembly 44 are each mounted to an object of interest, namely in this instance, to intravenous fluid tubing assembly 24. Nub assembly 44 includes substrate body 48 and substrate connectors 50A, 50B on opposing ends of the substrate body. Openable ends 64 of nubs 52 are mounted to the substrate body with the openable ends facing toward the embraced portion of the tubing assembly, thus toward luer connector 42.

Mounting apertures 84 extend through each of the substrate connectors. A slit 126 extends from each of the apertures 84 to the remote edges of the respective substrate connectors. In substrate connector 50A, the slit extends to a side edge of the substrate. In substrate connector 50B, the slit extends to remote end 47B of the substrate. Given the flexibility of substrate 46 at substrate connectors 50A, 50B, a remote end of the slit at the edge of the substrate can be urged against a length of tubing 40 whereupon the opposing edges of the respective slit 126 deflect thereby to provide a temporary opening through which tubing 40 can be moved into the respective aperture 84 such that the respective tubing becomes surrounded by the aperture, and the slit resiliently closes on itself. The tubing is thus held within the aperture until such time as the user desires to remove the nub assembly from the tubing assembly.

Intravenous fluid tubing assembly 24 represents a wide variety of such tubing assemblies, and shows a portion of the tubing assembly which includes a female luer connector 42. A first substrate connector 50A on a first end of the nub assembly is mounted to a first length of tubing 40A, and a second substrate connector 50B is mounted to a second length of tubing 40B. The first and second lengths of tubing are connected to, and extend from, the luer connector, whereby the luer connector is located generally between the opposing ends of the nub assembly.

When the user desires to remove the nub assembly from the tubing, the substrate is urged against the tubing at the inner edge of the respective slit at the respective aperture 84. The slit again resiliently opens up, and the tubing is removed from the substrate connector through the slit opening.

The slit can, of course, proceed in any direction from aperture 84 toward an edge of the substrate, whether laterally or longitudinally, so long as the slit does not interfere with the nubs as mounted on the substrate.

With the nub assembly mounted to the intravenous fluid tubing assembly as illustrated in FIG. 20, the individual disinfecting nubs are positioned at the anticipated location of use to disinfect luer connector 42. When the luer connector needs to be disinfected, the nubs are readily available at the use site. The user peels one of the nubs away from the substrate, which exposes the disinfectant carrier. The user then uses the exposed disinfectant carrier to disinfect, cleanse the luer connector or other medical access port at the use location. If the luer connector is to be maintained in a disinfected condition, the nub can be mounted on the luer connector and left mounted on the luer connector until such time as the luer connector is to be again used, or until uses of the luer connector are terminated. If the luer connector is not being maintained in a disinfected condition, the now-used nub is discarded. As desired in the disinfection process, the user can first squeeze the nub to thus expel a portion of the disinfectant carrier and disinfectant material as illustrated in FIG. 16.

When the luer connector again needs to be disinfected, the user selects a second different nub, peels that second nub away from the substrate and uses the second nub to disinfect the luer connector, with or without squeezing of the nub as desired.

In some embodiments, rather than peeling the nub away from the substrate, the openable end of the nub is sealed to the substrate with a relatively stronger bond, and lines of weakness 140 are formed in the substrate, and the openable ends of the respective nub housings are registered with the lines of weakness in the substrate. Such lines of weakness 140 are shown as dashed lines in the substrate in FIG. 20. Given the lines of weakness, when connector 42 is to be disinfected, cleansed, a nub is selected, the substrate is slid as necessary along the lengths of the tubings which extend through apertures 84 until the selected nub is over the connector. The luer connector is then pushed through the lines of weakness underlying the selected nub, thus breaking the lines of weakness and exposing the disinfectant carrier. The luer connector can then be disinfected using the disinfectant material in the nub. In some instances, the luer connector is first withdrawn from the nub, the nub is squeezed to expel a portion of the disinfectant carrier and the expelled portion is used to disinfect the medical access connector or the lines of weakness can be breached by squeezing the nub as in FIG. 16. The luer connector can, as in other embodiments, be left inside the nub if disinfection maintenance is the desired result. If the luer connector is to be used immediately, the luer connector is separated from the nub and the syringe or other medication carrier is connected to the luer connector and administered to the patient through the intravenous fluid tubing assembly as needed.

FIG. 20A illustrates mounting the nub assembly to the tubing assembly at both ends of the nub assembly, as in FIG. 20, but using hook and loop fasteners 54A, 54B at each end of the nub assembly in place of the slits and apertures, to wrap around the tubing as suggested by arrows 148.

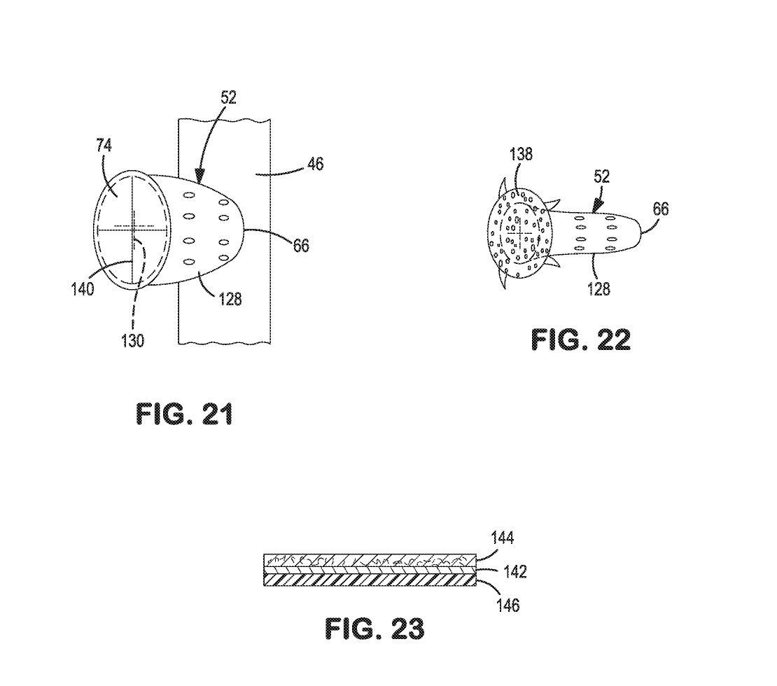

In some embodiments, illustrated in FIG. 21, the nub is mounted to the substrate at the dosed end of the nub housing, still with the nubs desirably, but not necessarily, facing toward the embraced portion of the tubing assembly. In such embodiments, a separate seal tab 74 is mounted to the openable end of each nub. The nub can always be opened by peeling the seal tab away from the openable end of the nub housing. Where the seal tab has lines of weakness 140 as shown in solid lines in FIG. 21, the disinfectant carrier can also be exposed and made available by breaching the lines of weakness. As suggested in FIG. 15, the lines of weakness can be breached by merely squeezing the nub housing at the housing sidewalls. In the alternative, the lines of weakness can be breached by pushing a luer connector or other medical access port into the nub through the lines of weakness. In either case, where the nub housing is positioned at the location of use as in FIG. 20, the nub need not be removed from the substrate before the nub is used. Rather, the luer connector is simply pushed into the openable end of the nub, namely at the tab bearing the lines of weakness. By leaving the nub attached to the substrate during use of the nub, there is no risk of dropping the nub, and there are fewer items of waste to discard when the medical procedure has been concluded.

FIG. 21 also shows, in dashed outline crossing lines of cut, invagination in the disinfectant carrier.

FIG. 22 is more of an end view of the nub after the lines of weakness in the tab have been breached, and after the nub has thereafter been squeezed to expel a portion 138 of the disinfectant carrier.

Material selection for substrate body 48 depends to some extent on how nub housing 62 is connected to the substrate. For example, where the openable end of the nub housing is secured to substrate body 48 as in FIGS. 4, 10, and 20, at least the surface layer of substrate body 48 is selected for its compatibility with being sealed to a respective layer of housing 62. With respect to FIGS. 4 and 10, the seal should be peelable such that the nub can be peeled away from the substrate body when the user needs to use a respective nub. With respect to FIG. 20, the seal should be non-peelable so the nub does not become separated from the substrate body while the lines of weakness are being penetrated and the nub is being used for cleansing the respective medical access port.

Where the nub housing extends through an aperture 56 as in FIGS. 5 and 6, substrate body 48 should be relatively non-extensible such that the substrate body securely holds the nubs/housings in apertures 56 until such time as the user chooses to release, remove a respective nub from the substrate. Further, the layer of the substrate which interfaces with sealing tip 82 should be compatible with holding the sealing tip fixedly attached to the substrate while the nub is being withdrawn through the respective aperture 56.

Where closed end 66 of the housing is secured to the substrate body, as in FIGS. 15 and 21, at least a surface layer of the substrate body is selected for its compatibility with being sealed, adhered to the outer layer of housing 62.

Those skilled in the art will be able to select suitable materials for substrate body 48 once the material for the housing, and the method of mounting the nub to the substrate body, are known. Exemplary, but not limiting, examples of materials which can be considered for use in and as substrate body 48 include polyethylenes, polypropylenes, polyethylene terephthalates, polyvinyl chlorides, and nylons.

Materials for a sealing tab over a disinfecting cap are well known, and typically include layers of metal foil, paper, and sealant, and may include adhesive layers on opposing sides of the metal foil layer. The invention here contemplates such materials for seal tabs 74, as well as seal tabs which are entirely composed of polymeric materials which are capable of retaining the disinfecting material inside the nub for a suitable shelf life.

Where the nubs are expected to be used only for scrubbing e.g. medication vials, the diameter, cross-section of the openable end of the housing can be any size which can allow for expelling a suitable scrubbing portion of the disinfectant carrier as at FIG. 16, and thus need not be sized to match any dimension of any particular medical access port. By contrast, where the nubs are expected to be used by inserting the port to be disinfected into the nub, as at FIGS. 17-19, the diameter, cross-section of the openable ends of the housings needs to be greater than the greatest diameter, cross-section of the ends of the ports which are to be disinfected.

Nubs of the invention have a housing profile which is ergonomically shaped for ease of holding, for squeezing progressing from the closed end toward openable end to facilitate expelling a portion of the disinfectant carrier. Such housing profiles can include protruding or recessed dimples, bulges, ridges or the like. In order that such nubs provide an effective amount of disinfectant material in a compact size, any recesses are substantially smaller than the curvature of a typical adult finger.

Having thus described the invention, what is claimed is:

1. A disinfecting nub assembly for assisting in preventing healthcare-associated infections, said disinfecting nub assembly, having an assembly length, and comprising:
(a) a substrate, comprising a substrate body, said substrate body having opposing surfaces, and opposing first and second ends, and a substrate body length between said first and second ends;
(b) a plurality of disinfecting nubs mounted to said substrate body, a given said disinfecting nub comprising a housing, said housing having an openable end and a closed end, and sidewalls extending between the openable end and the closed end, thus to define a nub cavity inside said housing, a disinfectant carrier being disposed in the nub cavity; and
(c) first and second substrate connectors associated with the opposing ends of said substrate body, said first and second substrate connectors having connector lengths extending away from the respective ends of said substrate body, said first and second substrate connectors cooperating with each other such that, when said first and second substrate connectors are wrapped at least part way around an object, said first and second substrate connectors can be connected to each other, thereby to mount said disinfecting nub assembly to such object.

2. A disinfecting nub assembly as in claim 1 wherein the connection of said first and second connectors to each other can be adjusted thus to facilitate utility of said disinfecting nub assembly relative to such object.

3. A disinfecting nub assembly as in claim 1 wherein said substrate connectors comprise at least one of
(i) a spring clip, or
(ii) hook and loop fasteners, or
(iii) male and female lock elements wherein said male lock element is inserted into said female lock element and subsequently locked to said female lock element by at least one of
A. rotation of said male and female lock elements relative to each other or
B. lateral movement of a male lock finger relative to said female lock element, or
(iv) wherein said second connector comprises at least one aperture extending through said substrate, or
(v) an aperture through at least one of said substrate connectors and a slit extending from such aperture to an outside edge of the respective said substrate connector.

4. A disinfecting nub assembly as in claim 1 wherein, when said disinfecting nub assembly is mounted to an object such that the object limits movement of said substrate body, the given disinfecting nub can be grasped and removed from said substrate against restraint of the object, by a worker using only one hand to so remove said nub from said substrate.

5. A disinfecting nub assembly as in claim 1, said disinfectant carrier occupying substantially all of the nub cavity while providing void spaces within said disinfectant carrier for accommodating a disinfectant material therein.

6. A disinfecting nub assembly as in claim 1, a disinfectant material being disposed in said disinfectant carrier, thus wetting said disinfectant carrier, and wherein said nub, when peeled away from said substrate body, can be squeezed to expel a portion of said disinfectant carrier from the nub cavity whereupon a user can use said expelled portion to scrub, and thereby to disinfect, a medical access port.

7. A disinfecting nub assembly as in claim 1, a given said nub extending through an aperture in said substrate, a disinfectant material being disposed in said disinfectant carrier, a sealing tab covering, and sealing, the openable end of the given said nub, said sealing tab comprising a cover portion which covers and releasably seals the openable end of the given said nub, and an overhang portion, extending from said cover portion, said overhang portion including a tether which is affixed to an adjacent portion of said substrate such that a user can grasp the given said nub and remove the respective nub from the corresponding aperture in said substrate while peeling the openable end of the respective said nub away from said sealing tab, thereby to expose said disinfectant carrier and said disinfectant material, while said tether retains said sealing tab affixed to said substrate.

8. A disinfecting nub assembly as in claim 1 wherein said housing is rigid and performs as a cap adapted to be removably mounted on a medical access port until such time as such medical access port is to be used to transfer a medical product.

9. A disinfecting nub assembly as in claim 6, said sidewalls of said housing tapering inwardly from the openable end toward the closed end whereby a cross-section of the nub cavity at the closed end has a smaller area than a cross-section of the nub cavity at the openable end, the tapering of said sidewalls facilitating expelling said disinfectant carrier from the cavity by the squeezing of said nub.

10. A disinfecting nub assembly as in claim 1, a disinfectant material being disposed in said disinfectant carrier, thus wetting said disinfectant carrier, and wherein a medical access port, on an intravenous fluid tubing assembly being used on a patient, can be inserted into the openable end of said housing and said housing subsequently squeezed about a longitudinal axis of said nub, and at least one of said nub and the medical access port rotated relative to the other while maintaining the squeezing, thus to apply a tangential scrubbing force to respective surfaces of the medical access port.

11. A disinfecting nub assembly as in claim 10 wherein said housing is made of material which embodies sufficient dead fold properties that, after such squeezing is relaxed, the dead fold properties of said housing are effective to retain such medical access port in said nub through a remainder of the use of such tubing assembly on such patient.

12. A disinfecting system comprising said disinfecting nub assembly as in claim 1, mounted to an appendage of a healthcare worker.

13. A disinfecting system comprising said disinfecting nub assembly as in claim 2, mounted to an appendage of a healthcare worker.

14. A disinfecting system comprising said disinfecting nub assembly as in claim 4, mounted to an appendage of a healthcare worker.

15. A disinfecting system comprising said disinfecting nub assembly as in claim 6, mounted to an appendage of a healthcare worker.

16. A disinfecting system comprising said disinfecting nub assembly as in claim 10, mounted to an appendage of a healthcare worker.

17. A disinfecting nub assembly for assisting in preventing healthcare-associated infections, said disinfecting nub assembly, and comprising:
(a) a substrate, comprising a substrate body, said substrate body having opposing surfaces, and opposing first and second ends, and a substrate body length between said first and second ends;
(b) a plurality of disinfecting nubs mounted to said substrate body, a given said disinfecting nub comprising a housing, said housing having an openable end and a closed end, and sidewalls extending between the openable end and the closed end, thus to define a nub cavity inside said housing, a disinfectant carrier being disposed in the nub cavity; and
(c) first and second substrate connectors associated with the opposing ends of said substrate body, said substrate connectors having connector lengths extending away from the respective ends of said substrate body, said first and second substrate connectors including tethers, said tethers being adapted and configured to tether spaced locations of said disinfecting nub assembly, proximate the opposing ends of said substrate to a given object.

18. A disinfecting nub assembly as in claim 17, said tethers, when said disinfecting nub assembly is tethered to a such object, being loosely mounted to such object so as to accommodate sliding movement of said tethers, and thus said disinfecting nub assembly, along a length of such object.

19. A disinfecting nub assembly as in claim 17, wherein said first connector comprises at least one of
(i) a hook which engages about such object, or
(ii) an aperture through at least one of said substrate connectors and a slit extending from such aperture to an outside edge of the respective said substrate connector, or
(iii) a spring clip, or
(iv) wherein a remote end of at least one of said first and second substrate connectors can be extended about such object and, while so extended about such object, connecting to a relatively proximal portion of the same respective said substrate connector using hook and loop fasteners, thus to form a tether loop extending entirely about the respective object, or
(v) wherein at least one of said first and second substrate connectors comprises at least one aperture extending through the proximal portion of the respective said tether.

20. A disinfecting nub assembly as in claim 17, a disinfectant material being disposed in said disinfectant carrier, and wherein a closure is sealed over the openable end of a given said disinfecting nub, thus to close off the openable end of the respective disinfecting nub, said closure comprising a line of weakness therein.

21. A disinfecting nub assembly as in claim 14, a disinfectant material being disposed in said disinfectant carrier, thus wetting said disinfectant carrier, and wherein said nub, when said disinfectant carrier is exposed at the openable end of said housing, can be squeezed to expel a portion of said disinfectant carrier from the nub cavity whereupon a user can use said expelled portion to scrub, and thereby to disinfect, a medical access port.

22. A disinfecting nub assembly as in claim 17, a given said nub extending through an aperture in said substrate, a sealing tab, comprising a cover portion, covering and releasably sealing the openable end of the given said nub, and an overhang portion, extending from said cover portion, said overhang portion including a tab tether affixed to an adjacent portion of said substrate such that a user can grasp the given said nub and remove the respective nub from the corresponding aperture in said substrate while peeling the openable end of the given said nub away from said sealing tab, thereby to expose said disinfectant carrier and said disinfectant material, while said tether retains said sealing tab affixed to said substrate.

23. A disinfecting nub assembly as in claim 17 wherein said housing is rigid and performs as a cap adapted to be removably mounted on a medical access port until such time as such medical access port is to be used to transfer a medical product.

24. A disinfecting nub assembly as in claim 17, said sidewalls of said housing tapering inwardly from the openable end toward the closed end whereby a cross-section of the nub cavity at the closed end has a smaller area than a cross-section of the nub cavity at the openable end, the tapering of said sidewalls facilitating expelling said disinfectant carrier from the cavity by squeezing said nub.

25. A disinfecting nub assembly as in claim 17, a disinfectant material being disposed in said disinfectant carrier, thus wetting said disinfectant carrier, and wherein a medical access port, on an intravenous fluid tubing assembly being used on a patient, can be inserted into the openable end of said housing and said housing subsequently squeezed about a longitudinal axis of said nub, and at least one of said nub and the medical access port rotated relative to the other while maintaining the squeezing, thus to apply a tangential scrubbing force to respective surfaces of the medical access port.

26. A disinfecting nub assembly as in claim 25 wherein said housing is made of material which embodies sufficient dead fold properties that, after such squeezing is relaxed, the dead fold properties of said housing are effective to retain such medical access port in said nub through a remainder of the use of such tubing assembly on such patient.

27. A disinfecting system comprising said disinfecting nub assembly as in claim 17 wherein said first and second substrate connectors are tethered to an intravenous tubing assembly, and wherein said disinfecting nub assembly, while so tethered to such intravenous tubing assembly, can slide freely along a length of such intravenous tubing assembly.

28. A disinfecting system comprising said disinfecting nub assembly as in claim 19 wherein said first and second substrate connectors are tethered to an intravenous tubing assembly, and wherein said disinfecting nub assembly, while so tethered to such intravenous tubing assembly, can slide freely along a length of such intravenous tubing assembly.

29. A disinfecting nub assembly for assisting in preventing healthcare-associated infections, said disinfecting nub assembly, having an assembly length, and comprising:
(a) a substrate, comprising a substrate body, said substrate body having opposing surfaces, and opposing first and second ends, and a substrate body length between said first and second ends;
(b) a plurality of disinfecting nubs mounted to said substrate body, a given said disinfecting nub comprising a housing, said housing having an openable end and a closed end, and sidewalls extending between the openable end and the closed end, thus to define a nub cavity inside said housing, a disinfectant carrier being disposed in the nub cavity, said disinfectant carrier being adapted to carry a disinfectant material therein; and
(c) a substrate extension extending from the first end of said substrate body, said substrate extension being adapted to mount said disinfecting nub assembly to an article of clothing, or a clothing appurtenance.

30. A disinfecting nub assembly as in claim 29 wherein said substrate extension is relatively more rigid than said substrate body and wherein said substrate extension extends in a generally U-shaped configuration, thereby to define a mounting cavity for receiving an edge of such article of clothing or clothing appurtenance.

31. A disinfecting nub assembly as in claim 29 wherein, when said substrate extension is mounted to such article of clothing or clothing appurtenance, said substrate body extends downwardly, drawn by gravity, from a joinder of said substrate body and said substrate extension.

32. A disinfecting nub assembly as in claim 29 wherein said substrate extension is devoid of any such disinfecting nubs.

33. A disinfecting nub assembly as in claim 29 wherein a first portion of said substrate extension, when displaced from a second different portion of said substrate extension, is resiliently biased against the second different portion of said substrate extension and exerts a resilient force against such article of clothing or clothing appurtenance in the mounting cavity.

34. A disinfecting nub assembly as in claim 29 wherein, when said disinfecting nub assembly is mounted to a user's clothing or clothing appurtenance, such mounting limits movement of said substrate body such that a given disinfecting nub can be grasped and removed from said substrate against restraint by such clothing or clothing appurtenance, such user using only one hand to so remove said nub from said substrate.

35. A disinfecting nub assembly as in claim 29, a disinfectant material being disposed in said disinfectant carrier, thus wetting the disinfectant carrier, and wherein a given said nub, when peeled away from said substrate body, can be squeezed to expel a portion of said disinfectant carrier from the nub cavity whereupon a user can use said expelled portion to scrub, and thereby to disinfect, a medical access port.

36. A disinfecting nub assembly as in claim 29, a given said nub extending through an aperture in said substrate, a disinfectant material being disposed in said disinfectant carrier, a sealing tab covering, and sealing, the openable end of the given said nub, and an overhang portion, extending from said cover portion, said overhang portion including a tether which is affixed to an adjacent portion of said substrate such that a user can grasp the given said nub and remove the respective nub from the corresponding aperture in said substrate while peeling the openable end of the respective said nub away from said sealing tab, thereby to expose said disinfectant carrier and said disinfectant material, while said tether retains said sealing tab affixed to said substrate.

37. A disinfecting nub assembly as in claim 29 wherein said housing is rigid and performs as a cap adapted to be removably mounted on a medical access port until such time as such medical access port is to be used to transfer a medical product.

38. A disinfecting nub assembly as in claim 29, said sidewalls of said housing tapering inwardly from the openable end toward the closed end whereby a cross-section of the nub cavity at the closed end has a smaller area than a cross-section of the nub cavity at the openable end, the tapering of said sidewalls facilitating expelling said disinfectant carrier from the cavity by the squeezing of said nub.

39. A disinfecting system comprising said disinfecting nub assembly as in claim 29, mounted to an article of clothing, or a clothing appurtenance, of a user.

40. A disinfecting nub assembly as in claim 29, a disinfectant material being carried in said disinfectant carrier, thus wetting said disinfectant carrier, and wherein a medical access port, on an intravenous fluid tubing assembly being used on a patient, can be inserted into the openable end of said housing and said housing subsequently squeezed about a longitudinal axis of said nub, and at least one of said nub and the medical access port rotated relative to the other while maintaining the squeezing, thus to apply a tangential scrubbing force to respective surfaces of the medical access port.

41. A disinfecting nub assembly as in claim 40 wherein said housing is made of material which embodies sufficient dead fold properties that, after such squeezing is relaxed, the dead fold properties of said housing are effective to retain such medical access port in said nub through a remainder of the use of such tubing assembly on such patient.

42. A disinfecting nub for assisting in preventing healthcare-associated infections in a medical environment, said disinfecting nub comprising:
(a) a housing having an openable end and a closed end, and sidewalls extending between the openable end and the closed end, thus to define a nub cavity inside said housing, said housing being manually deflectable toward a longitudinal axis which extends from the openable end to the closed end, to substantially collapse said nub housing;
(b) a disinfectant carrier disposed in the nub cavity;
(c) a disinfectant material disposed in said disinfectant carrier in said nub cavity, thus wetting said disinfectant carrier; and
(d) a seal closing the openable end of said housing,
wherein a medical access port, on an intravenous fluid tubing assembly being used on a patient, can be inserted into the openable end of said housing and subsequently squeezed about a longitudinal axis of said nub, and at least one of said nub and said medical access port rotated relative to the other while maintaining such squeezing, thus to simultaneously apply a tangential scrubbing force to substantially all respective surfaces of such medical access port.

43. A disinfecting nub as in claim 42 wherein said housing is made of materials which embody sufficient dead fold properties that, after such squeezing is released, the dead fold properties of said housing are effective to retain such medical access port in said nub through a remainder of the use of such tubing assembly on such patient.

44. A disinfecting nub as in claim 42, said sidewalls of said housing tapering inwardly from the openable end toward the closed end, a cross-section of the nub cavity near the closed end having a smaller area than a cross-section of the nub cavity at the openable end, while both such cross-sections have a generally common profile.

45. A disinfecting nub as in claim 44 wherein said sidewalls have nominal taper angle, from the openable end to the closed end, of about 5 degrees to about 25 degrees relative to the longitudinal central axis, and wherein the taper angle is relatively constant about a circumference of said nub housing.

46. A disinfecting nub as in claim 42 wherein said sidewalls of said housing have thicknesses of about 3 mils to about 45 mils in facilitating the squeezing of said nub sidewalls.

47. A disinfecting nub as in claim 42, said housing being made from a composite material comprising a polymeric layer, a metal foil layer, and a paper layer, said metal foil layer being between said polymeric layer and said paper layer.

48. A method of disinfecting a medical access port, comprising:
(a) mounting, on a user's body, a disinfecting nub assembly comprising
(i) a substrate, including a substrate body, and opposing first and second ends, and a substrate body length between the first and second ends,
(ii) a plurality of disinfecting nubs mounted to the substrate body, a given disinfecting nub comprising a housing, the housing having an openable end and a closed end, and sidewalls extending between the openable end and the closed end, thus to define a nub cavity inside the housing, a disinfectant carrier being disposed in the nub cavity, a disinfectant material being disposed in the disinfectant carrier, thus wetting the disinfectant carrier, and
(iii) first and second substrate connectors associated with opposing ends of the substrate body, the first and second substrate connectors having connector lengths extending away from the respective ends of the substrate body;
the mounting on the user's body comprising wrapping the first and second substrate connectors about a portion of such user's body and connecting the first and second substrate connectors to each other, thereby to mount the disinfecting nub assembly to the respective portion of the user's body;
(b) releasing a nub from the substrate and opening the respective nub at the openable end of the respective nub, thereby exposing the disinfectant carrier and the respective disinfectant material; and
(c) applying the disinfectant carrier, and thus the disinfectant material, to the medical access port, thus to disinfect the respective medical access port.

49. A method as in claim 48, further comprising adjusting the connection of the first and second substrate connectors to each other thereby to facilitate utility of the disinfecting nub assembly while mounted to the respective portion of the user's body.

50. A method as in claim 48 wherein the connecting of the first and second substrate connectors to each other comprises at least one of
(i) connecting hook and loop fasteners, or
(ii) connecting male and female lock elements wherein the male lock element is inserted into the female lock element and subsequently locked to the female lock element by at least one of rotation of the male and female lock elements relative to each other or by lateral movement of a male lock element member relative to the female lock element, or
(iii) connecting the first substrate connector to the second connector through at least one aperture extending through the substrate, or
(iv) connecting at least one of the first and second connectors to an aperture which extends through at least one of the substrate connectors and a slit extending from such aperture to an outside edge of the respective substrate connector.

51. A method as in claim 48 comprising mounting the disinfecting nub assembly to the user's body such that the mounting limits movement of the substrate body such that the user can grasp the nub and release the nub from the substrate using only one hand.

52. A method as in claim 48, further comprising, during a medical procedure, and with the disinfecting nub assembly mounted on the user's body, the user moving from a first location proximate an intravenous tubing assembly being used on a patient being treated, to a second location, displaced from the first location, proximate a supply source which provides a plurality of potentially useful medications, in medication containers and, while proximate the supply source, accessing a first disinfecting nub at the disinfecting nub assembly mounted to the user's body, and opening and using the accessed first disinfecting nub to disinfect a medical access port on a such medication container at the supply source, and drawing a medication through the so-disinfected medical access port.

53. A method as in claim 52, further comprising the user subsequently moving from the second location proximate the supply source to the first location proximate the intravenous tubing assembly being used on the patient being treated, accessing a second different disinfecting nub at the disinfecting nub assembly mounted to the user's body, and opening and using the second nub to disinfect a second medical access port on such intravenous tubing assembly using the second different disinfecting nub.

54. A method as in claim 48, further comprising releasing the nub from the substrate, subsequently squeezing the nub and thereby deflecting the sidewalls of the disinfecting nub inwardly, and substantially collapsing the nub housing, causing a portion of the disinfectant carrier to protrude from the openable end of the housing, and thereafter using the protruding portion of the disinfectant carrier to scrub the medical access port.

55. A method as in claim 48, further comprising inserting a medical access port, on an intravenous fluid tubing assembly being used on a patient, into the openable end of a such nub on the disinfecting nub assembly, and subsequently squeezing the nub about a longitudinal axis of the nub, and rotating at least one of the nub and the medical access port relative to the other while maintaining the squeezing, thus applying a tangential scrubbing force to the respective surfaces of the medical access port.

56. A method as in claim 55, further comprising releasing the squeezing, and wherein the housing is made of material which embodies sufficient dead fold properties that, after such squeezing is released, the dead fold properties of the housing retain the medical access port in the nub cavity through a remainder of the use of the tubing assembly on such patient.

57. A method of disinfecting a medical access port, comprising:
(a) mounting, on a user's body, a disinfecting nub assembly comprising
(i) a substrate, including a substrate body, and opposing first and second ends, and a substrate body length between the first and second ends,
(ii) a plurality of disinfecting nubs mounted to the substrate body, a given disinfecting nub comprising a housing, the housing having an openable end and a closed end, and sidewalls extending between the openable end and the closed end, thus to define a nub cavity inside the housing, a disinfectant carrier being disposed in the nub cavity, a disinfectant material being carried in the disinfectant carrier, a closure being sealed over the openable end of the housing, and
(iii) a substrate extension extending from the first end of the substrate body, the substrate extension being adapted to mount the disinfecting nub assembly to an article of clothing, or a clothing appurtenance,
the mounting on the user's body comprising mounting the adapter to an article of clothing or a clothing appurtenance on the user's body;
(b) releasing a given nub from the substrate and opening the respective nub at the openable end of the respective nub, thereby exposing the disinfectant carrier and the respective disinfectant material carried therein; and
(c) applying the disinfectant carrier, and thus the disinfectant material, to the medical access port, thus to disinfect the respective medical access port.

58. A method as in claim 57, further comprising, after releasing the nub from the substrate, squeezing the nub and thereby deflecting the sidewalls of the disinfecting nub inwardly, and substantially collapsing the nub housing, causing a portion of the disinfectant carrier to protrude from the openable end of the housing, and thereafter using the protruding portion of the disinfectant carrier to scrub the medical access port.

59. A method as in claim 57, the substrate extension being generally rigid and non-extensible such that the substrate extension accommodates releasing the nub from the substrate without substantial extension or bending of the substrate extension.

60. A method as in claim 57 wherein the substrate extension is relatively more rigid than the substrate body and wherein the substrate extension extends in a generally U-shaped configuration, thereby to define a mounting cavity receiving such article of clothing or clothing appurtenance.

61. A method as in claim 60 wherein a first portion of the substrate extension, when displaced from a second different portion of said substrate extension, is resiliently biased against the second portion of the substrate extension, and exerts a resilient force against such clothing or clothing appurtenance in the mounting cavity.

62. A method as in claim 57, further comprising, after releasing the given nub from the substrate and exposing the disinfectant carrier, squeezing the nub, and thereby deflecting sidewalls of the nub inwardly and substantially collapsing the nub housing and thereby causing a portion of the disinfectant carrier to protrude from the housing, and thereafter using the protruding portion of the disinfectant carrier, and the liquid carried therein, to scrub the medical access port.

63. A method as in claim 57, further comprising inserting a medical access port, on an intravenous fluid tubing assembly being used on a patient, into the openable end of a such nub on the disinfecting nub assembly, and subsequently squeezing the nub about a longitudinal axis of the nub, and rotating at least one of the nub and the medical access port relative to the other while maintaining the squeezing, thus applying a tangential scrubbing force to the respective surfaces of the medical access port.

64. A method as in claim 63, further comprising releasing the squeezing, and wherein the housing is made of material which embodies sufficient dead fold properties that, after such squeezing is released, the dead fold properties of the housing retain the medical access port in the nub cavity through a remainder of the use of the tubing assembly on such patient.

65. A method of disinfecting a medical access port, comprising:
(a) mounting, to an object, a disinfecting nub assembly, the disinfecting nub assembly comprising
(i) a substrate, including a substrate body, and opposing first and second ends, and a substrate body length between the first and second ends,
(ii) a plurality of disinfecting nubs mounted to the substrate body, a given disinfecting nub comprising a housing, the housing having an openable end and a closed end, and sidewalls extending between the openable end and the closed end, thus to define a nub cavity inside the housing, a disinfectant carrier being disposed in the nub cavity, a disinfectant material being disposed in the disinfectant carrier, and a closure sealed over the openable end of the housing,
(iii) first and second substrate connectors associated with the opposing ends of the substrate body, the substrate connectors having connector lengths extending away from the respective ends of the substrate body, the first and second substrate connectors including tethers, the tethers being adapted and configured to tether spaced locations of the disinfecting nub assembly, proximate the opposing ends of the substrate, to a given object,
the mounting of the disinfecting nub assembly to the object comprising at least one of
(I) mounting the first substrate connector to the object at a first location on the object and mounting the second substrate connector to the object at a second location on the object displaced from the first location, or
(II) wrapping the first substrate connector about the object at a first location on the object and thereafter securing a first remote portion of the first substrate connector to a first proximal portion of the disinfecting nub assembly, and wrapping the second substrate connector about the object at a second location on the object, spaced from the first location, and thereafter securing a second remote portion of the second substrate connector to a second proximal portion of the disinfecting nub assembly;
(b) opening the respective nub at the openable end of the nub, thereby exposing the disinfectant carrier and the respective disinfectant material; and
(c) applying the disinfectant carrier, and thus the disinfectant material, to a such medical access port, thus to disinfect the respective medical access port.

66. A method as in claim 65, the object comprising an intravenous tubing assembly, the mounting comprising wrapping the first substrate connector about the intravenous tubing assembly, and thereafter securing a remote portion of the first substrate connector to a first proximal portion of the disinfecting nub assembly, and wrapping the second substrate connector about the same intravenous tubing assembly and thereafter securing a remote portion of the second substrate connector to a second proximal portion of the disinfecting nub assembly.

67. A method as in claim 66, comprising wrapping the first and second substrate connectors loosely about the intravenous tubing assembly such that the disinfecting nub assembly can slide freely along at least a portion of a length of the intravenous tubing assembly.

68. A method as in claim 67 comprising positioning the disinfecting nub assembly along the length of the intravenous tubing assembly such that a medical access port on the intravenous tubing assembly is between the first and second substrate connectors.

69. A method as in claim 68, further comprising orienting the disinfecting nub assembly such that the sealed ends of the disinfecting nubs face generally toward the medical access port on the intravenous tubing assembly.

70. A method as in claim 69 wherein closures are sealed over the openable ends of respective ones of the disinfecting nubs, and wherein respective closures comprise a line of weakness, the lines of weakness optionally being disposed away from end edges of the sidewalls of the respective nub housings, the method comprising grasping a such disinfecting nub and breaching the line of weakness, thereby exposing the disinfectant carrier, and applying the disinfectant carrier, bearing the disinfectant material, to the medical access port to thereby disinfect the respective medical access port.

71. A method as in claim 70 including using the medical access port to breach the line of weakness on the closure of a respective disinfecting nub.

72. A method as in claim 71 including, after using the medical access port to breach the line of weakness, squeezing the nub sidewalls inwardly and thereby expelling a portion of the disinfectant carrier from the openable end of the housing, and subsequently using the so expelled portion of the disinfectant carrier, and the carried disinfectant material carried therein, to scrub the respective medical access port.

73. A method as in claim 69, comprising using the medical access port to breach the line of weakness on a given such disinfecting nub, subsequently squeezing the nub sidewalls inwardly and thereby expelling a portion of the disinfectant carrier from the openable end of the housing, and subsequently using the so expelled portion of the disinfectant carrier, and the carried disinfectant material, to scrub, and thereby disinfect, the medical access port on the intravenous tubing assembly, all while the respective disinfecting nub remains attached to the substrate and the disinfecting nub assembly remains mounted to the intravenous tubing assembly.

74. A method as in claim 73, the given disinfecting nub comprising a first disinfecting nub, the method further comprising breaching the line of weakness on a second such disinfecting nub which is mounted on the substrate, subsequently squeezing the sidewalls on the second disinfecting nub inwardly and thereby expelling a second portion of disinfectant carrier, including the disinfectant material carried therein, from the openable end of the second nub housing, and subsequently using the so expelled second portion of disinfectant carrier from the second disinfecting nub to scrub, and thereby disinfect, the same medical access port on the intravenous tubing assembly a second time, all while the second disinfecting nub remains attached to the substrate and the disinfecting nub assembly remains mounted to the same intravenous tubing.

75. A method as in claim 65, further comprising inserting a medical access port, on an intravenous fluid tubing assembly being used on a patient, into the openable end of a such nub on the disinfecting nub assembly, and subsequently squeezing the nub about a longitudinal axis of the nub, and rotating at least one of the nub and the medical access port relative to the other while maintaining the squeezing, thus applying a tangential scrubbing force to the respective surfaces of the medical access port.

76. A method as in claim 75, further comprising releasing the squeezing, and wherein the housing is made of material which embodies sufficient dead fold properties that, after such squeezing is released, the dead fold properties of the housing retain the medical access port in the nub cavity through a remainder of the use of the tubing assembly on such patient.

77. A method of disinfecting a medical access port, comprising:
(a) accessing a disinfecting nub, the disinfecting nub comprising
(i) a housing having a closed end and an openable end, and sidewalls extending between the openable end and the closed end, thus to define a nub cavity inside the housing, the housing being manually deflectable toward a longitudinal axis which extends from the openable end to the closed end, to thereby substantially collapse the nub housing,
(ii) a disinfectant carrier disposed in the nub cavity,
(iii) a disinfectant material disposed in the disinfectant carrier in the nub cavity, and
(iv) a closure sealed over the openable end of the housing;
(b) inserting a medical access port, on an intravenous fluid tubing assembly being used on a patient, into the openable end of a such nub on the disinfecting nub assembly; and
(c) subsequently squeezing the nub about the longitudinal axis of the nub and rotating at least one of the nub and the medical access port about the other while maintaining the squeezing, thus applying a tangential scrubbing force to the respective surfaces of the respective medical access port.

78. A method as I claim 77, further comprising releasing the squeezing, and wherein the housing is made of material which embodies sufficient dead fold properties that, after such squeezing, the dead fold properties of the housing retain the medical access port in the nub through a remainder of the use of the tubing assembly on such patient.

79. A method as in claim 77, the closure sealed over the openable end of the housing comprising a line of weakness, the method comprising using the medical access port to breach the line of weakness.

80. A method as in claim 79, further comprising, after breaching the closure at the line of weakness using the medical access port, withdrawing the medical access port from the disinfecting nub, squeezing the nub housing and thereby causing a portion of the disinfectant carrier, and a respective portion of the disinfectant material carried therein, to protrude from the openable end of the housing, and then using the protruding portion of the disinfectant carrier, containing the disinfectant material, to scrub, and thereby disinfect, the medical access port.

81. A method as in claim 77, further comprising the sidewalls of the housing tapering inwardly from the openable end toward the closed end whereby a cross-section of the nub cavity proximate the closed end has a smaller area than a cross-section of the nub cavity at the openable end, and thereby facilitating the disinfectant carrier protruding from the housing when the housing is squeezed.

82. A method as in claim 81 wherein the sidewalls have a nominal taper angle, from the openable end to the closed end, of about 5 degrees to about 25 degrees relative to the longitudinal central axis of the disinfecting nub.

83. A method as in claim 81 wherein the sidewalls of the housing have thicknesses of about 3 mils to about 45 mils, thereby accommodating the squeezing, and inward deflecting, of the housing sidewalls.

84. A method as in claim 78 wherein the housing comprises a composite material comprising a heat sealable layer, a metal foil layer, and a paper layer, the metal foil layer being between the heat sealable layer and the paper layer.

85. A method as in claim 77, the method further comprising, after squeezing the nub and thereby deflecting the sidewalls of the disinfecting nub inwardly and causing the portion of the disinfectant carrier to protrude from the openable end of the housing, at least partially releasing the squeezing of the nub sidewalls and inserting the medical access port into the openable end of the housing, thereby mounting the disinfecting nub onto the medical access port.
